(19) United States
(12) Patent Application Publication
(10) Pub. No.: US 2015/0218286 A1
(43) Pub. Date: Aug. 6, 2015

(54) Methods of Treating Pediatric Acute Lymphoblastic Leukemia with an Anti-CD22 Immunotoxin

(71) Applicant: MedImmune, LLC, Gaithersburg, MD (US)

(72) Inventors: Alan S. WAYNE, Los Angeles, CA (US); Ira Pastan, Potomac, MD (US)

(21) Appl. No.: 14/553,875

(22) Filed: Nov. 25, 2014

Related U.S. Application Data
(63) Continuation of application No. 13/207,856, filed on Aug. 11, 2011, now abandoned.
(60) Provisional application No. 61/372,813, filed on Aug. 11, 2010.

(51) Int. Cl. C07K 16/30; A61K 47/02; A61K 39/395; A61K 45/06; C07K 16/28; C07K 14/21

(52) U.S. Cl. CPC C07K 16/3061; C07K 16/2803; C07K 14/21; A61K 39/39558; A61K 45/06; A61K 47/02; C07K 2317/622; C07K 2317/624; C07K 2317/92; C07K 2317/73; C07K 2319/55; A61K 2039/505

(57) ABSTRACT

The present invention provides methods for the treatment of acute lymphoblastic leukemia (ALL) in pediatric patients using an anti-CD22 immunotoxin. The methods disclosed comprise administering to a pediatric patient in need of that treatment an effective dose of a recombinant immunotoxin comprising a variable light (V_{L}) chain linked to a variable heavy (V_{H}) which is genetically fused to a therapeutic moiety comprising a Pseudomonas exotoxin A PE38 fragment. The recombinant immunotoxin specifically binds CD22 thereby inhibiting the growth of CD22-expressing (CD22⁺) ALL cancer cells.

### CROSS REFERENCE TO RELATED APPLICATIONS

The present application is a continuation of U.S. Utility patent application Ser. No. 13/207,856, filed Aug. 11, 2011. The present application claims priority benefit to U.S. Provisional Application Ser. No. 61/372,813, filed Aug. 11, 2010, which is incorporated herein by reference in its entirety.

### REFERENCE TO SEQUENCE LISTING SUBMITTED ELECTRONICALLY

The content of the electronically submitted sequence listing (Name: 2943_{—}0030002_Sequence_Listing.txt; Size: 16,755 bytes; and Date of Creation: Apr. 6, 2015) filed with the application is incorporated herein by reference in its entirety.

### BACKGROUND OF THE INVENTION

1. Field of the Invention

The present invention provides methods and compositions for treating acute lymphoblastic leukemia in pediatric patients with an anti-CD22 immunotoxin.

2. Background Art

Hematological malignancies are a major public health problem. It has been estimated that in the year 2000, more than 50,000 new cases of non-Hodgkin's lymphoma and more than 30,000 new cases of leukemia occurred in the United States (Greenlee, R. T. et al., CA Cancer J. Clin., 50:7-33 (2000)) and more than 45,000 deaths were expected from these diseases. Many more patients live with chronic disease-related morbidity. Unfortunately, in a high percentage of patients, conventional therapies are not able to induce long term complete remissions.

Acute lymphoblastic leukemia (“ALL”) is the most common pediatric cancer. Each year, 2,500 to 3,000 children and adolescents in the United States are diagnosed with B-lineage acute lymphoblastic leukemia. 75% to 80% of children with B-precursor ALL achieve long-term relapse-free survival with current treatments that incorporate combination chemotherapy and central nervous system prophylactic therapy. However, the outlook remains guarded for individuals with certain high-risk features at diagnosis and for those who relapse (Gaynon, P. S, Br. J. Haematol. 131:579-587 (2005); Gloekler Ries, L. A., NIH Pub. No. 99-4649. p. 165-170 (1999)). Additionally, current therapies carry the risk of treatment associated morbidity and mortality (Pui, C.-H., et al., N. Engl. J. Med. 349:640-649 (2003); Oeffinger, K. C., et al., N. Engl. J. Med. 355:1572-1582 (2006)). Thus, novel approaches that can overcome chemotherapy resistance and decrease non-specific toxicities are needed to improve the outcome for children with hematologic malignancies.

In the past several years immunotoxins have been developed as an alternative therapeutic approach to treat these malignancies. Immunotoxins are usually composed of an antibody chemically conjugated to a plant or a bacterial toxin. The antibody binds to the antigen expressed on the target cell and the toxin is internalized causing cell death by arresting protein synthesis and inducing apoptosis (Brinkmann, U. Mol. Med. Today, 2:439-446 (1996)).

One antigen that has been used as an immunotoxin target is CD22, a lineage-restricted B cell antigen expressed in 60-70% of B cell lymphomas and leukemias. CD22 is not present on the cell surface in the early stages of B cell development and is not expressed on stem cells (Tedder, T. F., et al., Annu. Rev. Immunol., 5:481-504 (1997)). Clinical trials have been conducted with an immunotoxin containing an anti-CD22 antibody, RFB4, or its Fab fragment, coupled to deglycosylated ricin A. In these trials, substantial clinical responses have been observed; however, severe and in certain cases fatal, vascular leak syndrome was dose limiting (Sausville, E. A., et al., Blood, 85:3457-3465 (1995); Amlot, P. L., et al., Blood, 82:2624-2633 (1993); Vitetta, E. S., et al., Cancer Res., 51:4052-4058 (1991)).

As an alternative approach, the RFB4 antibody has been used to make a recombinant immunotoxin in which the Fv fragment in a single chain form is fused to a 38 kDa truncated form of Pseudomonas exotoxin A (PE38). PE38 contains the translocating and ADP ribosylating domains of PE but not the cell-binding portion (Hwang, J., et al., Cell, 48:129-136 (1987)). RFB4(Fv)-PE38 is cytotoxic towards CD22-positive cells (Mansfield, E., et al., Biochem. Soc. Trans., 25:709-714 (1997)). To stabilize the single chain Fv immunotoxin and to make it more suitable for clinical development, cysteine residues were engineered into framework regions of the V_{H} and V_{L} (Mansfield, E. et al., Blood 90:2020-2026 (1997)) generating the molecule RFB4(dsFv)-PE38 (also known as “BL22” or “CAT-3888”).

CAT-3888 is able to kill leukemic cells from patients and induced complete remissions in mice bearing lymphoma xenografts (Kreitman, R. J., et al., Clin. Cancer Res., 6:1476-1487 (2000); Kreitman, R. J., at al., Int. J. Cancer, 81:148-155 (1999)), CAT-3888 was evaluated in a phase I clinical trial at the National Cancer Institute in patients with hematological malignancies. Sixteen patients with purine analogue resistant hairy cell leukemia were treated with CAT-3888 and eleven (86%) achieved complete remissions. CAT-3888 appears to work well on malignancies, such as HCL, which express CD22 in high density. It showed much less activity, however, in CALL, in which the cells have lower levels of expression of CD22.

Since CAT-3888 has been shown to be capable of achieving complete remission in some malignancies, a Phase I clinical trial evaluated the side effects and best dose of CAT-3888 immunotoxin in treating ALL in pediatric patients (Wayne, A. S., et al., Clin. Cancer Res., 16(6):1894-1903 (2010)). Transient clinical activity, such as reduction in circulating blasts, normalized blood counts, and decreased blast infiltration of bone marrow, were observed in 70% of the patients. However, no remissions were observed. Thus, a need remains for therapies capable of achieving complete remissions in ALL patients refractory to chemotherapy.

CAT-8015 (also known as “HA22”) is an affinity-matured form of CAT-3888. In CAT-8015, residues SSY in the complementarity determining region (“CDR”) 3 of the RFB4 antibody variable region heavy chain (“V_{H}”) were mutated to THW. Compared to its parental RFB4 antibody, CAT-8015 has a 5 to 10-fold increase in cytotoxic activity on various CD22-positive cell lines and is up to 50 times more cytotoxic to cells from patients with CLL and HCL (Salvatore, G., et al., Clin. Cancer Res., 8(4):995-1002 (2002)).

### BRIEF SUMMARY OF THE INVENTION

The present invention provides methods for the treatment of ALL in patients, e.g., pediatric patients using an anti-CD22 immunotoxin. In this regard, the invention provides a method of treating ALL in pediatric patients comprising administering to a pediatric patient in need of that treatment an effective dose of a recombinant immunotoxin, wherein the immunotoxin comprises a variable light (V_{L}) chain and a variable heavy (V_{H}), wherein said V_{H} chain is genetically fused to a therapeutic moiety comprising a PE38 Pseudomonas exotoxin A fragment. The recombinant immunotoxin can be a full-length antibody molecule, a single chain Fv (“scFv”), a disulfide stabilized Fv (“dsFv”), an Fab, or an F(ab′). The recombinant immunotoxin specifically binds CD22 thereby inhibiting the growth of CD22-expressing (CD22⁺) cancer cells. The V_{L} chain comprises the sequence of antibody RFB4, and the V_{H} chain comprises the sequence of antibody RFB4, but in which residues SSY in CDR3 of the RFB4 antibody variable region heavy chain (“V_{H}”) were mutated to THW. The immunotoxin can further comprise a linker interposed between the immunotoxin V_{H} chain and the therapeutic moiety. In one embodiment, the sequence of the linker is KASGG. In one embodiment, the linker is interposed between the carboxy-terminal amino acid of the V_{H} chain and the amino-terminal amino acid of a PE38 Pseudomonas exotoxin A polypeptide. In one embodiment, the immunotoxin is CAT-8015.

The treatments of the present invention can be administered to pediatric patients suffering from ALL, relapsed ALL, or refractory ALL. In some embodiments, the treatment of the present invention can be administered to adult patients suffering from ALL, relapsed ALL, or refractory ALL. In some embodiments, the immunotoxin is administered in a combination therapy. The immunotoxin can be administered to the patient during or after treatment with at least one single agent or multi-agent combination treatment regimen. In some embodiments, the immunotoxin is administered to a patient who has received a stem cell transplant or a bone marrow transplant prior to the treatment with the immunotoxin. In yet further embodiments, the immunotoxin is administered to a patient who has received radiation therapy, either as conditioning for bone marrow transplant or stem cell transplant or as a therapy.

In another group of embodiments, the immunotoxin used in the methods of treatment of the present invention is formulated with a pharmaceutically acceptable carrier, adjuvant, diluent, excipient, or any combinations thereof. In some embodiments, the formulation is lyophilized. In some embodiments, the immunotoxin is formulated at a concentration from about 0.5 mg/mL to about 2.5 mg/mL. In some embodiments, the immunotoxin is formulated at a concentration of about 1.0 mg/mL, or about 1.1 mg/mL, or about 12 mg/mL, or about 1.3 mg/mL, or about 1.4 mg/mL, or about 1.5 mg/mL. In one embodiment, the immunotoxin is formulated at a concentration of about 0.7 mg/mL. The immunotoxin can be formulated as a solution for injection comprising sodium chloride, potassium dihydrogen phosphate, disodium hydrogen phosphate, and sodium hydroxide. In one embodiment, the formulated immunotoxin is CAT-8015.

In still further embodiments, the present invention provides for treatments wherein the immunotoxin is administered to a patient, e.g., a pediatric patient in need thereof in combination with at least one therapeutic agent select from the group consisting of an antibody or derivative thereof, a cytotoxic agent, a drug, a toxin, a radionuclide, an immunomodulator, a photoactive therapeutic agent, a radiosensitizing agent, and a hormone.

In yet further embodiments, the immunotoxin is administered as an intravenous injection. The intravenous injection can be, for example, an intravenous infusion (IV infusion). In an embodiment, the IV infusion is administered over a period of about 30 minutes.

In some embodiments, the inhibition of the growth of CD22⁺ cancer cells following the administration of the immunotoxin results in complete remission (complete response), improvement in response, lowering of leukemia burden, or a combination thereof. In certain embodiments, the inhibition of the growth of CD22⁺ cancer cells following the administration of the immunotoxin results in complete remission

In some embodiments, the present invention provides methods for treating ALL in pediatric patients wherein the range of immunotoxin dose administered to the pediatric patient in need of treatment is from about 5 μg/kg to about 30 μg/kg. The immunotoxin dose can be, for example, about 5 μg/kg, about 10 μg/kg, about 20 μg/kg, about 30 μg/kg, about 40 μg/kg, about 50 μg/kg, about 60 μg/kg, about 70 μg/kg, about 80 μg/kg, about 90 μg/kg, or about 100 μg/kg.

In some embodiments, the immunotoxin can be administered to a patient in need of treatment for one or more treatment cycles. In yet further embodiments, the patient can be treated with escalating doses of the immunotoxin.

The method of the invention includes administering to a patient, e.g., a pediatric patient in need thereof a therapeutically effective amount of an immunotoxin, such as CAT-8015, such that an effective exposure is provided in a pediatric patient, for example as measured by the pharmacokinetic parameters Cₘₐₓ, T_{1/2}, AUC_{0 ∞}, Clearance, etc.

In some embodiments, the administration of immunotoxin achieves an arithmetic peak plasma concentration (Cₘₐₓ) of immunotoxin in a range of from about 311 μg/mL to about 586 μg/mL. In some embodiments, the median of the arithmetic peak plasma concentrations (Cₘₐₓ) of immunotoxin derived from a population of patients after the administration of immunotoxin is about 516 μg/mL. In yet further embodiments, the Cₘₐₓ of immunotoxin derived from a population of patients after the administration of immunotoxin is greater than about 360 μg/mL.

In some embodiments, the immunotoxin biological half-life (T_{1/2}) after the administration of immunotoxin is in a range of from about 36 minutes to about 138 minutes. In some embodiments, the median of T_{1/2} values derived from a population of patients after the administration of immunotoxin is about 60 minutes. In yet further embodiments, the median of T_{1/2} values derived from a population of patients after the administration of immunotoxin is lower than about 100 minutes.

In some embodiments, a plot of the plasma concentration of immunotoxin versus time following the administration of immunotoxin yields an arithmetic area under the curve from time zero to infinity (AUC_{0 ∞}) for immunotoxin in a range of from about 5.8 μg*min/mL to about 33.2 μg*min/mL. In some embodiments, the median of the AUC_{0 ∞} values derived from a population of patients after the administration of immunotoxin is about 14.5 μg*min/mL. In yet further embodiments, the median of the AUC_{0 ∞} values derived from a population of patients after the administration of immunotoxin is lower than about 50 μg*min/mL.

In some embodiments, the immunotoxin clearance rate (Cl) following the administration of immunotoxin is in a range from about 15,100 mL/kg/hour to about 85,200 mL/kg/hour. In some embodiments, the median of Cl values derived from a population of patients after the administration of immunotoxin is about 36,400 mL/kg/hour.

In yet further embodiments, the immunotoxin has a binding affinity for CD22 with a dissociation constant (K_{d}) of less than 80 nM. In some embodiments, the immunotoxin has a binding affinity for CD22 with a dissociation constant (K_{d}) of about 6 nM.

In yet another group of embodiments, the present invention provides methods for the treatment of ALL in pediatric patients wherein the inhibition of the growth of CD22⁺ cancer cells is caused by immunotoxin-induced cytotoxicity. In some embodiments, the immunotoxin-induced cytotoxicity causes an increase in cellular apoptosis.

In some embodiments, the present invention provides methods for the treatment of ALL in pediatric patients wherein the in vitro cytoxicity of the CAT-8015 immunotoxin is significantly greater than the level of cytotoxicity of the CAT-3888 immunotoxin, wherein the cytoxicity (LC₅₀) is measured as the concentration of immunotoxin that kills 50% of a population of viable B-lineage ALL cells. In some embodiments, the ratio between the level of cytoxicity of the CAT-8015 immunotoxin and the level of cytotoxicity of the CAT-3888 immunotoxin, LC_{50 CAT-8015}/LC_{50 CAT-3888}, is at least 1.5.

### BRIEF DESCRIPTIONS OF THE DRAWINGS

FIG. 1A shows a scatter plot of the percentage of viable ALL cells from relapsed and newly diagnosed patients remaining after 72 h incubation with 500 ng/mL CAT-8015 (HA22). FIG. 1B shows a scatter plot of the 50% lethal CAT-8015 concentration (LC₅₀) that results after treatment of ALL cells in samples from relapsed and newly diagnosed patients with CAT-8015 (HA22).

FIG. 2 shows a scatter plot of the percentage of dead cells after incubation with 500 ng/mL CAT-8015 (HA22) versus the number of CD22 sites per cell.

FIG. 3 shows the blood counts in one of the pediatric patients with chemotherapy-refractory ALL, treated with CAT-8015 after two treatment cycles with 30 μg/kg dosage. Counts were falling due to progressive bone marrow infiltration at the time of trial enrollment. Arrows represent doses of CAT-8015. Absolute neutrophil count (ANC) and platelet count are represented as black and grey lines, respectively.

FIG. 4A shows a hematoxylin and eosin (H&E) stain (100× magnification) of bone marrow aspirate from an 8 year old patient with chemotherapy-refractory ALL prior to treatment with CAT-8015. FIG. 4B shows a hematoxylin and eosin (H&E) stain (100× magnification) of bone marrow aspirate from the same patient after treatment cycle 1 with CAT-8015 (10 μg/kg, six doses administered every other day).

FIGS. 5A, 5B, 5C and 5D show Wright-Giemsa stains (1000× magnification) of bone marrow aspirate samples from an 11 year old patient with multiply recurrent ALL who had undergone two prior stem cell transplants, showing the response after treatment with 20 μg/kg CAT-8015. FIG. 5A shows pre-treatment cells. FIG. 5B shows cells on day 15 of treatment cycle 1. FIG. 5C shows cells on day 14 of treatment cycle 2. FIG. 5D shows cells on day 21 of treatment cycle 3.

FIGS. 6A, 6B, 6C and 6D show Tdt stains (40× magnification) of bone marrow biopsy samples from an 11 year old patient with multiply recurrent ALL who had undergone two prior stem cell transplants showing the response after treatment with 20 μg/kg CAT-8015. FIG. 6A shows pre-treatment bone marrow. FIG. 6B shows bone marrow on day 15 of treatment cycle 1. FIG. 6C shows bone marrow on day 14 of treatment cycle 2. FIG. 6D shows bone marrow on day 21 of treatment cycle 3.

FIGS. 7A, 7B, and 7C show Wright-Giemsa stains (1000× magnification) of bone marrow aspirate samples from a 14 year old patient with chemotherapy-refractory ALL who had undergone a prior stem cell transplant showing the response after treatment with 30 μg/kg CAT-8015, FIG. 7A shows pre-treatment cells. FIG. 7B shows cells on day 14 of treatment cycle 1. FIG. 7C shows cells on day 14 of treatment cycle 2.

FIGS. 8A, 8B, and 8C show Tdt stains (40× magnification) of bone marrow biopsy samples from a 14 year old patient with chemotherapy-refractory ALL who had undergone a prior stein cell transplant showing the response after treatment with 30 μg/kg CAT-8015. FIG. 8A shows pre-treatment bone marrow. FIG. 8B shows bone marrow on day 14 of treatment cycle 1. FIG. 8C shows bone marrow on day 14 of treatment cycle 2.

FIG. 9 shows cytotoxicity curves after treatment of blasts from ALL patients with CAT-8015 (HA22; light gray, right curve) and its variants HA22-LR (black, center center curve) and HA-22-LR-8X (dark gray, left curve).

FIGS. 10A, 10B, 10C, 10D, 10E and 10F show the response of blasts from ALL patients after treatment with the CAT-8015 variant HA22-LR. FIG. 10A shows a scatter plot of the percentage of viable cells from relapsed and newly diagnosed patients after incubation with 500 ng/mL HA22-LR. FIG. 10C shows a scatter plot of the percentage of viable cells in whole blood and bone marrow samples after incubation with 500 ng/mL HA22-LR. FIG. 10E shows a scatter plot of the percentage of cell viability following incubation with 500 ng/mL HA22-LR versus the percentage of cell viability following incubation with 10 μmol/L dexamethasone in samples from newly diagnosed patients. FIG. 10B shows a scatter plot of the IC_{50'}s of cells from relapsed and newly diagnosed patients after incubation with HA22-LR. FIG. 10D shows a scatter plot of the percentage of viable cells following incubation with 500 ng/mL HA22-LR as a function of the number of CD22 sites/cell. FIG. 10F shows a scatter plot of the percentage of cell viability following incubation with 500 ng/mL HA22-LR versus the percentage of cell viability following incubation with 10 μmol/L dexamethasone in samples from relapsed patients.

FIGS. 11A, 11B, 11C, 11D, 11E, and 11F show the response of blasts from ALL patients after treatment with the CAT-8015 variant HA-LR-8X. FIG. 11A shows a scatter plot of the percentage of viable cells from relapsed and newly diagnosed patients after incubation with 500 ng/mL HA22-LR-8X. FIG. 11C shows a scatter plot of the percentage of viable cells in whole blood and bone marrow samples after incubation with 500 ng/mL HA22-LR-8X. FIG. 11E shows a scatter plot of the percentage of cell viability following incubation with 500 ng/mL HA22-LR-8X versus the percentage of cell viability following incubation with 10 μmol/L dexamethasone in samples from newly diagnosed patients. FIG. 11B shows a scatter plot of the IC_{50'}s of cells from relapsed and newly diagnosed patients after incubation with HA22-LR-8X. FIG. 11D shows a scatter plot of the percentage of viable cells following incubation with 500 ng/mL HA22-LR-8X as a function of the number of CD22 sites/cell. FIG. 11F shows a scatter plot of the percentage of cell viability following incubation with 500 ng/mL HA22-LR-8X versus the percentage of cell viability following incubation with 10 μmol/L dexamethasone in samples from relapsed patients.

### DETAILED DESCRIPTION OF THE INVENTION

The invention provides methods for the treatment of pediatric acute lymphoblastic leukemia with the CAT-8015 (HA22) immunotoxin. In one embodiment, pediatric patients with relapsed or refractory CD22⁺ B-lineage ALL are treated with CAT-8015 immunotoxin administered at doses of 5, 10, 20, or 30 μg/kg every-other-day for 6 doses every 21 days for up to 6 cycles. In contrast to previous trials in which the CAT-3888 immunotoxin was used, complete remission was observed when patients were treated with CAT-8015. Details of the methods are provided herein.

### Definitions

“CD22” refers to a lineage-restricted B cell antigen belonging to the Ig superfamily. It is expressed in 60-70% of B cell lymphomas and leukemias and is not present on the cell surface in early stages of B cell development or on stem cells. See, e.g. Vaickus, et al., Crit. Rev. Oncol/Hematol. 11:267-297 (1991).

As used herein, the term “anti-CD22” in reference to an antibody, refers to an antibody that specifically binds CD22 and includes reference to an antibody which is generated against CD22. In some embodiments, the CD22 is a primate CD22 such as human CD22. In one embodiment, the antibody is generated against human CD22 synthesized by a non-primate mammal after introduction into the animal of cDNA which encodes human CD22.

The terms “polypeptide,” “peptide,” “protein,” and “protein fragment” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function similarly to the naturally occurring amino acids.

As used herein, “recombinant” includes reference to a protein produced using cells that do not have, in their native state, an endogenous copy of the DNA able to express the protein. The cells produce the recombinant protein because they have been genetically altered by the introduction of the appropriate isolated nucleic acid sequence.

As used herein, the “antibody” and “immunoglobulin” are used interchangeably in the broadest sense and include monoclonal antibodies (e.g., full length or intact monoclonal antibodies), polyclonal antibodies, multivalent antibodies, multispecific antibodies (e.g., bispecific antibodies so long as they exhibit the desired biological activity) and antibody fragments as described herein. Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The terms “constant” and “variable” are used functionally.

The constant domain of the light chain is aligned with the first constant domain of heavy chain, and the light chain variable domain is aligned with the variable domain of the heavy chain. Particular amino acid residues are believed to form an interface between the light and heavy chain variable domains (Clothia, et al., J. Mol. Biol. 186, 651-66 (1985); Novotny and Haber, Proc. Natl. Acad. Sci. USA 82, 4592-4596 (1985)). Five human immunoglobulin classes are defined on the basis of their heavy chain composition, and are named IgG, IgM, IgA, IgE, and IgD. The IgG-class and IgA-class antibodies are further divided into subclasses, namely, IgG1, IgG2, IgG3, and IgG4, and IgA1 and IgA2. The heavy chains in IgG, IgA, and IgD antibodies have three constant region domains, that are designated CH1, CH2, and CH3, and the heavy chains in IgM and IgE antibodies have four constant region domains, CH1, CH2, CH3, and CH4. Thus, heavy chains have one variable region and three or four constant regions. Immunoglobulin structure and function are reviewed, for example, in Harlow et al., Eds., Antibodies: A Laboratory Manual, Chapter 14, Cold Spring Harbor Laboratory, Cold Spring Harbor (1988).

References to “V_{H}” or a “VH” refer to the variable region of an immunoglobulin heavy chain, including an Fv, scFv, dsFv or Fab.

References to “V_{L}” or a “VL” refer to the variable region of an immunoglobulin light chain, including an Fv, scFv, dsFv, or Fab.

The term “antibody fragment” refers to a portion of an intact antibody and refers to the antigenic determining variable regions of an intact antibody. Examples of antibody fragments include, but are not limited to Fab, Fab′, F(ab′)2, Fv and single chain Fv fragments, linear antibodies, single chain antibodies, and multispecific antibodies formed from antibody fragments.

The terms “single chain Fv” or “scFv” refers to an antibody in which the variable domains of the heavy chain and of the light chain of a traditional two chain antibody have been joined to form one chain. Terms include binding molecules which consist of one light chain variable domain (V_{L}) or portion thereof, and one heavy chain variable domain (V_{H}) or portion thereof, wherein each variable domain (or portion thereof) is derived from the same or different antibodies. scFv molecules typically comprise an scFv linker interposed between the V_{H} domain and the V_{L} domain. scFv molecules are known in the art and are described, in U.S. Pat. No. 5,892,019, Ho, et al., Gene 77:51-59 (1989); Bird, et al., Science 242:423-426 (1988); Pantoliano, et al., Biochemistry 30:10117-10125 (1991); Milenic, et al., Cancer Research 51:6363-6371 (1991); Takkinen, et al., Protein Engineering 4:837-841 (1991), all of which are hereby incorporated by reference in their entireties.

The term “linker” refers to molecule that covalently or non-covalently connects two or more molecules, thereby creating a larger complex consisting of all molecules including the linker molecule. The term “linker” comprises both polypeptide linkers, and non-polypeptide linkers.

The phrase “disulfide stabilized Fv” or “dsFv” refer to the variable region of an immunoglobulin in which V_{H} and V_{L} are synthesized as separate polypeptides and are joined by a disulfide bond between V_{H} and V_{L}. In the context of this invention, the cysteines which form the disulfide bond are within the framework regions of the antibody chains and serve to stabilize the conformation of the antibody. Typically, the antibody is engineered to introduce cysteines in the framework region at positions where the substitution will not interfere with antigen binding.

As used herein the term “disulfide bond” refers to the covalent bond formed between two sulfur atoms. The amino acid cysteine comprises a thiol group that can form a disulfide bond or bridge with a second thiol group.

“RFB4” refers to a mouse IgG1 monoclonal antibody that specifically binds to human CD22. RFB4 is commercially available under the name RFB4 from several sources, such as Southern. Biotechnology Associates, Inc. (Birmingham Ala.; Cat. No. 9360-01), Autogen Bioclear UK Ltd. (Calnc, Wilts, UK; at No. AB147), Axxora LLC. (San Diego, Calif.). RFB4 is highly specific for cells of the B lineage and has no detectable cross-reactivity with other normal cell types (Li et al., Cell. Immunol. 118:85-99 (1989). The heavy and light chains of RFB4 have been cloned (see, Mansfield et al., Blood 90:2020-2026 (1997)).

That an antibody “specifically binds” means that the antibody reacts or associates more frequently, more rapidly, with greater duration, with greater affinity, or with some combination of the above to an epitope than with alternative substances, including unrelated proteins. “Specifically binds” means, for instance, that an antibody binds to a protein with a K_{D} of at least about 0.1 mM, but more usually at least about 1 μM. “Specifically binds” means at times that an antibody binds to a protein at times with a K_{D} of at least about 0.1 μM or better, and at other times at least about 0.01 μM or better. Because of the sequence identity between homologous proteins in different species, specific binding can include an antibody that recognizes a tumor cell marker protein in more than one species.

The antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al, Proc. Natl. Acad. Sci. USA 81:6851-6855 (1984)).

Light and heavy chain variable regions, V_{H} and V_{L}, contain a “framework” region interrupted by three hypervariable regions, also called “complementarity-determining regions” or “CDRs”. The sequences of the framework regions of different light or heavy chains are relatively conserved within a species. The framework region of an antibody, that is the combined framework regions of the constituent light and heavy chains, serves to position and align the CDRs in three dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The terms “fusion protein” or “chimeric protein” or descriptions of a protein or polypeptide comprising two moieties that are “fused,” refer to a first amino acid sequence linked to a second amino acid sequence with which it is not naturally linked in nature. The amino acid sequences may normally exist in separate proteins that are brought together in the fusion polypeptide or they may normally exist in the same protein but are placed in a new arrangement in the fusion polypeptide. A fusion protein may be created, for example, by chemical synthesis, or by creating and translating a polynucleotide in which the peptide regions are encoded in the desired relationship. As used herein, the terms “linked,” “fused” or “fusion” are used interchangeably.

The term “immunoconjugate” or “conjugate” as used herein refers to a compound or a derivative thereof that is fused or linked to a cell binding agent (i.e., an anti-CD22 antibody or fragment thereof) and is defined by a generic formula: C-L-A, wherein C=cytotoxin, L=linker, and A=cell binding agent or anti-CD22 antibody or antibody fragment. Immunoconjugates can also be defined by the generic formula in reverse order: A-L-C. An “immunoconjugate” comprises a targeting portion, or moiety, such as an antibody or fragment thereof which retains antigen recognition capability, and an effector molecule, such as a therapeutic moiety or a detectable label.

An “immunotoxin” is an immunoconjugate in which the therapeutic moiety is a cytotoxin. A “targeting moiety” is the portion of an immunoconjugate intended to target the immunoconjugate to a cell of interest. Typically, the targeting moiety is an antibody, a scFv, a dsFv, an Fab, or an F(ab′)₂. The targeting moiety can also comprise, e.g., a Fab′, a Fd, V-NAR domain, an IgNar, an intrabody, an IgGΔCH2, a minibody, a F(ab′)₃, a tetrabody, a triabody, a diabody, a single-domain antibody, DVD-Ig, Fcab, mAb², a (scFv)₂, or a scFv-Fc.

A “toxic moiety” is the portion of a immunotoxin which renders the immunotoxin cytotoxic to cells of interest.

A “therapeutic moiety” is the portion of an immunoconjugate intended to act as a therapeutic agent and in some embodiments can be a “toxic moiety”.

The term “therapeutic agent” includes any number of compounds currently known or later developed to act as, e.g., anti-neoplastics, anti-inflammatories, cytokines, anti-infectives, enzyme activators or inhibitors, allosteric modifiers, antibiotics or other agents administered to induce a desired therapeutic effect in a patient. The therapeutic agent can also be a toxin or a radioisotope, where the therapeutic effect intended is, for example, the killing of a cancer cell.

The terms “effective amount” or “amount effective to” or “therapeutically effective amount” includes reference to a dosage of a therapeutic agent sufficient to produce a desired result, such as inhibiting cell protein synthesis by at least 50%, or killing the cell.

The term “in vivo” includes reference to inside the body of the organism from which the cell was obtained.

The terms “ex vivo” and “in vitro” mean outside the body of the organism from which the cell was obtained.

The terms “patient” or “pediatric patient” refers to a human subject from about 6 months of age to about 24 years of age suffering from ALL which has been specifically chosen to receive a therapeutic treatment.

The phrase “dissociation constant” refers to the affinity of an antibody for an antigen. Specificity of binding between an antibody and an antigen exists if the dissociation constant (K_{D}=1/Kₐ, where Kₐ is the affinity constant) of the antibody is <1 mM, for example, <100 nM, for example, <0.1 nM. Antibody molecules will typically have a K_{D} in the lower ranges. K_{D}=[Ab-Ag]/[Ab][Ag] where [Ab] is the concentration at equilibrium of the antibody, [Ag] is the concentration at equilibrium of the antigen and [Ab-Ag] is the concentration at equilibrium of the antibody-antigen complex. Typically, the binding interactions between antigen and antibody include reversible non-covalent associations such as electrostatic attraction, van der Waals forces and hydrogen bonds. This method of defining binding specificity applies to single heavy and/or light chains, CDRs, fusion proteins or fragments of heavy and/or light chains, that are specific for CD22 if they bind CD22 alone or in combination.

The term “maximum plasma concentration” (“Cₘₐₓ”) refers to the highest observed concentration of immunotoxin in plasma following administration of the immunotoxin to the patient.

The term “biological half-life” (“T_{1/2}”) is defined as the time required for the plasmatic concentration of immunotoxin to reach half of its original value.

The term “area under the curve” (“AUC”) is the area under the curve in a plot of the concentration of immunotoxin in plasma against time. AUC can be a measure of the integral of the instantaneous plasma concentrations (Cₚ) during a time interval and has the units of mass*time/volume. AUC is usually given for the time interval zero to infinity. Thus, as used herein “AUC_{0 ∞}” refers to an AUC from over an infinite time period.

The term “clearance” (“Cl”) refers to the volume of plasma cleared of the immunotoxin per unit time.

The disclosed values for the pharmacokinetic data generally apply to a population of patients treated according to the methods disclosed in the present invention, not to individual patients. Thus, any individual patient treated according to the disclosed methods will not necessarily achieve the desired pharmacokinetic parameters. However, when the methods of treatment of the present invention are administered to a sufficiently large population of pediatric patients, the pharmacokinetic parameters will approximately match the values disclosed herein.

The term “relapse” relates to the return of signs and symptoms of a disease after a patient has enjoyed a remission, e.g. after therapy such as chemotherapy and/or radiation therapy. In particular, the term “relapse” relates to the reappearance of cancer after a disease-free period. For example, after treatment a patient with cancer may go into remission with no sign or symptom of the tumor, remain in remission for some time, but then suffer a relapse that requires the patient to be treated once again for cancer.

The term “refractory” when used herein means that malignancies are generally resistant to treatment or cure. The present invention, where treatment of refractory cancers and the like is mentioned, is to be understood to encompass not only cancers where one or more chemotherapeutics have already failed during treatment of a patient, but also cancers that can be shown to be refractory by other means, e.g. biopsy and culture in the presence of chemotherapeutics.

As used herein, “a method of treating” a hematological malignancy such as ALL means that the disease and the symptoms associated with the disease are alleviated, reduced, cured, or placed in a state of remission.

As used herein, the term “favorable biological properties” includes biological properties other than the ability of an immunotoxin to inhibit the growth of CD22-expressing cancer cells and/or treat ALL, which enhance the ability of the immunotoxin to perform its intended function, e.g., to treat ALL. In one embodiment, the favorable biological properties is a pharmacokinetic profile. Examples of such parameters which can be used, include, but are not limited to Cₘₐₓ, AUC_{0 ∞}, T_{1/2}, and Cl. In a further embodiment, the favorable biological property is a target plasma concentration or a tarizet systemic exposure.

The term “lyophilized” refers to any composition or formulation at is prepared in dry form by rapid freezing and dehydration, in the frozen state under high vacuum.

“Lyophilizing” or “lyophilization” refers to a process of freezing and drying a solution.

As used in the present disclosure and claims, the singular forms “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety.

### CAT-8015 (HA22) Immunotoxin

CAT-8015 (“HA22”), described in detail international Patent Application Publication Nos. WO 98/41641 and WO2003/27135, and in Salvatore, G. et al., Clin. Cancer Res. 8(4):995-1002 (2002), all of which are incorporated by reference herein in their entireties, is an affinity-optimized recombinant immunotoxin protein composed of an antibody Fv fragment based on the murine anti-CD22 antibody RFB4 fused to a truncated form of the Pseudomonas exotoxin (PE) protein, PE38. The anti-CD22 Fv fragment consists of two domains, a V_{L} and a V_{H}, where the latter was modified to improve binding to the human CD22 target.

The CAT-8015 protein comprises two independent polypeptides, the V_{L} chain (SEQ ID NO:2), and the V_{H} chain, fused at the C-terminus to the PE38 domain (V_{H}-PE38) (SEQ ID NO:1). Other V_{L} and V_{H}-PE38 sequences useful in this invention are described in U.S. Pat. Nos. 7,541,034 and 7,355,012, and in U.S. Patent Application Publication No. 2007/0189962, all of which are incorporated by reference herein in their entireties. Both domains were designed to each contain engineered cysteine residues that permit formation of an intermolecular disulfide bond. This feature increases the stability of the fusion protein. Both polypeptides are expressed in E. coli cells and isolated from inclusion bodies.

Amino Acid Sequence of the V_{H} moiety (SEQ ID NO:1) of the V_{H}-PE38 subunit of CAT-8015:

MEVQLVESGGGLVKPGGSLKLSCAASGFAFSIYDMSWVRQTPEKCLEWVA
YISSGGGTTYYPDTVKGRFTISRDNAKNTLYLQMSSLKSEDTAMYYCARH
SGYGTHWGVLFAYWGQGTLVTVS

CAT-3888 differs from CAT-8015 in that three amino acids (Ser-Ser-Tyr; SSY) in CDR3 of CAT-3888 are mutated in CAT-8015 to Thr-Trp-His (TWH). The three mutated residues are shown underlined in SEQ ID NO:1, above.

Amino Acid Sequence of the Pseudomonas exotoxin PE38 moiety (SEQ ID NO: 2) of the V_{H}-PE38 subunit of CAT-8015:

PEGGSLAALTAHQACHLPLETFTRHRQPRGWEQLEQCGYPVQRLVALYLA
ARLSWNQVDQVIRNALASPGSGGDLGEAIREQPEQARLALTLAAAESERF
VRQGTGNDEAGAANGPADSGDALLERNYPTGAEFLGDGGDVSFSTRGTQN
WTVERLLQAHRQLEERGYVFVGYHGTFLEAAQSIVFGGVRARSQDLDAIW
RGFYIAGDPALAYGYAQDQEPDARGRIRNGALLRVYVPRSSLPGFYRTSL
TLAAPEAAGEVERLIGHPLPLRLDAITGPEEEGGRLETILGWPLAERTVV
IPSAIPTDPRNVGGDLDPSSIPDKEQAISALPDYASQPGKPPREDLK

Amino Acid Sequence of CAT-8015 V_{H}-PE38 Subunit (SEQ ID NO:3), including the five amino acid linker (KASGG; SEQ ID NO: 4; underlined) interposed between the V_{H} domain and the PE38 domain:

MEVQLVESGGGLVKPGGSLKLSCAASGFAFSIYDMSWVRQTPEKCLEWVA
YISSGGGTTYYPDTVKGRFTISRDNAKNTLYLQMSSLKSEDTAMYYCARH
SGYGTHWGVLFAYWGQGTLVTVSAKASGGPEGGSLAALTAHQACHLPLET
FTRHRQPRGWEQLEQCGYPVQRLVALYLAARLSWNQVDQVIRNALASPGS
GGDLGEAIREQPEQARLALTLAAAESERFVRQGTGNDEAGAANGPADSGD
ALLERNYPTGAEFLGDGGDVSFSTRGTQNWTVERLLQAHRQLEERGYVFV
GYHGTFLEAAQSIVFGGVRARSQDLDAIWRGFYIAGDPALAYGYAQDQEP
DARGRIRNGALLRVYVPRSSLPGFYRTSLTLAAPEAAGEVERLIGHPLPL
RLDAITGPEEEGGRLETILGWPLAERTVVIPSAIPTDPRNVGGDLDPSSI
PDKEQAISALPDYASQPGKPPREDLK

Amino Acid Sequence of the V_{L} Subunit (SEQ ID NO:5) of CAT-8015:

MDIQMTQTTSSLSASLGDRVTISCRASQDISNYLNWYQQKPDGTVKLLIY
YTSILHSGVPSRFSGSGSGTDYSLTISNLEQEDFATYFCQQGNTLPWTFG
CGTKLEIK

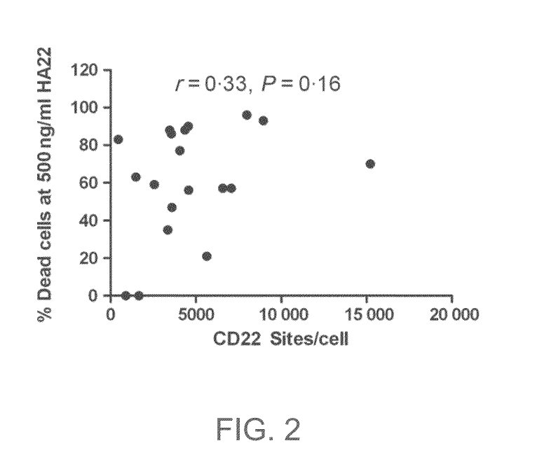

CAT-8015 was developed in order to improve upon the activity of CAT-3888, a closely related predecessor compound, in the treatment of a variety of CD22+ B-cell malignancies (Salvatore et al., 2002). CAT-8015 differs from CAT-3888 by three contiguous amino acids (CDR3 Ser-Ser-Tyr amino acids mutated to Thr-His-Trp), As a result of this change, the binding affinity of CAT-8015 for CD22 improved approximately 14-fold over CAT-3888, to a K_{d} of 6 nanomolar (nM) from 85 nM. The increased affinity resulted in a significant improvement in cytotoxic activity of CAT-8015, when compared with CAT-3888, against B-cell cancer cell lines, malignant cells isolated from patients with H+CL and CLL (Salvatore et al., 2002; Decker, T. et al., Blood 103:2718-2726 (2004)), and malignant cells isolated from patients with ALL (Mussai F., et al., Br. J. Hematol. 150:352-358 (2010)). CAT-3888 cytotoxic activity is related to the number of CD22 expressed on the cancer cell surface, while CAT-8015 toxicity is less dependent on the level of CD22 cell surface expression (FIG. 2).

### CAT-8015 (HA22) Immunotoxin Variants

CAT-8015 variants can be generated by fusing the V_{H} moiety of SEQ ID NO: 2 to alternative versions of the Pseudomonas exotoxin (see, e.g., Hansen, J. K., et al., Journal of Immunotherapy 33:297-301 (2010); Weldon, J. E., et al., Blood 113:3792-3800 (2009), which are hereby incorporated by reference in their entireties). Suitable variants of the Pseudomonas exotoxin include:

PE-LR
(SEQ. ID. NO: 6)
RHRQPRGWEQLPTGAEFLGDGGDVSFSTRGTQNWTVERLLQAHRQLEERG
YVFVGYHGTFLEAAQSIVFGGVRARSQDLDAIWRGFYIAGDPALAYGYAQ
DQEPDARGRIRNGALLRVYVPRSSLPGFYRTSLTLAAPEAAGEVERLIGH
PLPLRLDAITGPEEEGGRLETILGWPLAERTVVIPSAIPTDPRNVGGDLD
PSSIPDKEQAISALPDYASQPGKPPREDLK
PE-LR-6X
(SEQ. ID. NO: 7)
RHRQPRGWEQLPTGAEFLGDGGDVSFSTRGTQNWTVERLLQAHRQLEEGG
YVFVGYHGTFLEAAQSIVFGGVRARSQDLDAIWAGFYIAGDPALAYGYAQ
DQEPDAAGRIRNGALLRVYVPRSSLPGFYATSLTLAAPEAAGEVERLIGH
PLPLRLDAITGPEEAGGRLETILGWPLAERTVVIPSAIPTDPRNVGGDLD
PSSIPDSEQAISALPDYASQPGKPPREDLK
PE-LR-8X
(SEQ. ID. NO: 8)
RHRQPRGWEQLPTGAEFLGDGGAVSFSTRGTQNWTVERLLQAHRQLEEGG
YVFVGYHGTFLEAAQSIVFGGVRARSQDLDAIWAGFYIAGDPALAYGYAQ
DQEPDAAGAIRNGALLRVYVPRSSLPGFYATSLTLAAPEAAGEVERLIGH
PLPLRLDAITGPEEAGGRLETILGWPLAERTVVIPSAIPTDPRNVGGDLD
PSSIPDSEQAISALPDYASQPGKPPREDLK

Treatment of Pediatric ALL Patient with CAT-8015 (HA22)

The present invention provides methods for the treatment of acute lymphoblastic leukemia (ALL) in patients, e.g., pediatric patients, using an anti-CD22 immunotoxin. In this regard, the invention provides methods of treating ALL in pediatric patients comprising administering to a pediatric patient in need of that treatment an effective dose of a recombinant immunotoxin, wherein the immunotoxin comprises a variable light (V_{L}) chain and a variable heavy (V_{H}), wherein said V_{H} chain is genetically fused to a therapeutic moiety comprising a PE38 Pseudomonas exotoxin A fragment, and wherein the recombinant immunotoxin specifically binds CD22 thereby inhibiting the growth of CD22 cancer cells.

In certain embodiments, the recombinant immunotoxin is, e.g., a full length antibody molecule, a single chain Fv (“scFv”), a disulfide stabilized Fv (“dsFv”), an Fab, or an F(ab′). The recombinant immunotoxin specifically binds CD22 thereby inhibiting the growth of CD22-expressing (CD22⁺) cancer cells.

In some embodiments, the immunotoxin comprises a variable light (V_{L}) chain comprising SEQ ID NO: 5 and a variable heavy (V_{H}) chain comprising SEQ ID NO: 1, wherein said V_{H} chain is genetically fused to a therapeutic moiety comprising a PE38 Pseudomonas exotoxin A fragment. In some embodiments, the PE38 exotoxin fragment is SEQ ID NO: 2. In some embodiments the immunotoxin further comprises a linker interposed between the variable heavy (V_{H}) chain and the therapeutic moiety. This linker can be interposed between the immunotoxin variable heavy (V_{H}) chain and the therapeutic moiety. In some embodiments, the linker is interposed between the carboxy-terminal amino acid of the V_{H} chain and the amino-terminal amino acid of a PE38 Pseudomonas exotoxin A polypeptide. For example, the linker can comprise SEQ ID NO:4. In some embodiments, the V_{H}-PE38 Subunit of the immunotoxin comprises SEQ ID NO: 3. In one embodiment, the immunotoxin is CAT-8015.

The treatments of the present invention can be administered to pediatric patients suffering from ALL, relapsed ALL, or refractory ALL. In some embodiments, the treatments of the present invention can be administered to adult patients.

In some embodiments, the immunotoxin is administered in a combination therapy. The present invention provides for treatments wherein the immunotoxin is administered to a patient, e.g., a pediatric patient in need thereof, in combination with at least one therapeutic agent, for example, an antibody or derivative thereof, a cytotoxic agent, a drug, a toxin, a radionuclide, an immunomodulator, a photoactive therapeutic agent, a radiosensitizing agent, and a hormone.

The immunotoxin can be administered to the patient prior, during or after treatment with at least one single agent or multi-agent combination treatment regimen. Therapies that can be administered in combination with immunotoxins include, but are not limited to, surgical procedures (e.g., bone marrow transplantation), radiation therapy, chemotherapy, monoclonal antibody therapy, other immunotoxin therapy, small-molecule based cancer therapy, vaccine/immunotherapy-based cancer therapies, or other cancer therapy, where the additional cancer therapy is administered prior to, during, or subsequent to the immunotoxin therapy of the present invention. Thus, where the combined therapies comprise administration of immunotoxin in combination with administration of another therapeutic agent, as with, e.g., chemotherapy, radiation therapy, other anti-cancer immunotoxin therapy, anti-cancer antibody therapy, small molecule-based cancer therapy or vaccine/immunotherapy-based cancer therapy, the methods of the invention encompass co-administration using separate formulations or a single pharmaceutical formulation, or a consecutive administration in either order. Where the methods of the present invention comprise combined therapeutic regimens, these therapies can be given simultaneously (i.e., concurrently or within the same time frame as the other cancer therapy). Alternatively, the immunotoxin can be administered prior to or subsequent to the other cancer therapy. Sequential administration of the different cancer therapies can be performed regardless whether the treated pediatric patient responds to the first course of therapy to decrease the possibility of remission or relapse.

In some embodiments, the immunotoxin is administered to a patient, e.g., a pediatric patient who has received a stem cell transplant or a bone marrow transplant prior to the treatment with the immunotoxin. For example, the stem cell transplant can be an autologous stem cell transplant or an allogeneic stem cell transplant.

In yet further embodiments, the immunotoxin is administered to a pediatric patient who has received radiation therapy either as conditioning for bone marrow transplant or stem cell transplant or as a therapy.

The immunotoxins are administered at a concentration that is therapeutically effective to prevent or treat ALL. To accomplish this goal, the immunotoxins can be formulated using a variety of a pharmaceutically acceptable carriers, adjuvants, diluents, excipients, or any combinations thereof known in the art.

Typically, the immunotoxins are administered by injection, for example, by intravenous infusion (IV infusion). Intravenous administration can occur by infusion of a period of about 30 minutes. The infusion can be given over longer or shorter periods of time as required. The initial infusion can be given over a period of about 30 minutes, with subsequent infusions delivered over different time periods.

The method of the invention is performed using an immunotoxin formulated to be compatible with its intended route of administration. In some embodiments, the formulation is lyophilized. In some embodiments, the immunotoxin is formulated at concentrations from about 0.5 mg/mL to about 2.5 mg/mL. In one embodiment, the concentration of immunotoxin is about 0.7 mg/mL. The immunotoxin is formulated as a solution for injection comprising sodium chloride, potassium dihydrogen phosphate, disodium hydrogen phosphate, and sodium hydroxide. In one embodiment, the formulated immunotoxin is CAT-8015.

In some embodiments, the inhibition of the growth of CD22-expressing (CD22⁺) cancer cells following the administration of the immunotoxin results in complete remission (complete response), improvement in response, lowering of leukemia burden, or a combination thereof. In one embodiment, the inhibition of the growth of CD22⁺ cancer cells tumor following the administration of the immunotoxin results in complete remission.

The amount of immunotoxin to be administered is readily determined by one of ordinary skill in the art without undue experimentation. Factors influencing the mode of administration and the respective amount of immunotoxin include, but are not limited to, the severity of the disease, the history of the disease, and the age, height, weight, health, and physical condition of the individual undergoing therapy. Similarly, the amount of immunotoxin to be administered will be dependent upon the mode of administration and whether the patient will undergo a single dose or multiple doses of the immunotoxin. Generally, a higher dosage of immunotoxin is desired with increased weight of the pediatric patient undergoing therapy.

As one of skill in the art will understand, other factors will influence the ideal dose regimen in a particular case. Such factors can include, for example, the binding affinity and half-life of the immunotoxin, the degree of overexpression of CD22 in the patient, the desired steady-state immunotoxin concentration level, frequency of treatment, and the influence of other therapies used in combination with the treatment method of the invention.

In some embodiments, the present invention provides methods for treating ALL in pediatric patients wherein the range of immunotoxin dose administered to the pediatric patient in need of treatment is from about 1 μg/kg to about 50 μg/kg, for example, in the range of about 5 μg/kg to about 40 μg/kg. The immunotoxin dose can be, for example, about 5 μg/kg, about 10 μg/kg, about 20 μg/kg, about 30 μg/kg, about 40 μg/kg, about 50 μg/kg, about 60 μg/kg, about 70 μg/kg, about 80 μg/kg, about 90 μg/kg, or about 100 μg/kg.

Single or multiple administrations of the compositions can be administered depending on the dosage and frequency as required and tolerated by the patient. In any event, the composition should provide a sufficient quantity of the proteins of this invention to effectively treat the patient. Generally, the dose should be sufficient to treat or ameliorate symptoms or signs of disease without producing unacceptable toxicity to the patient. An effective amount of the compound is that which provides either subjective relief of a symptom(s) or an objectively identifiable improvement as noted by the clinician or other qualified observer. Typically, the frequency of dosing depends upon the pharmacokinetic parameters of the immunotoxin in the formulation used. Generally, the immunotoxin is administered until a dosage is reached that achieves the desired effect. The immunotoxin can therefore be administered as a single dose or multiple doses (at the same or different concentrations/dosages) over time, or as a continuous infusion. Further refinement in the dosage is routinely made. Appropriate dosages can he ascertained through use of the appropriate dose-response data.

In one embodiment of the invention, the method comprises the administration of multiple doses of immunotoxin. In some embodiments, the method comprises the administration of, e.g., 1, 2, 3, 4, 5, 6, 7, 8, 9, 10, 15, 20, or more therapeutically effective doses of a pharmaceutical composition comprising the immunotoxin. In one embodiment, the method comprises the administration of 6 doses. In some embodiments, the method comprises the administration of immunotoxin doses, for example, every day, every 2 days, every 3 days, etc. The range of time between doses can he greater than 3 days. In some embodiments, therapeutically effective doses of a pharmaceutical composition comprising the immunotoxin are administered every other day. The total dosage can he administered in a single or multiple infusions in a given day.

Treatment of a pediatric patient with a therapeutically effective amount of the immunotoxin can include a single treatment cycle a series of treatments cycles. The pediatric patient can be treated with immunotoxin for about 1 to 10 weeks. The range of time can be greater than 10 weeks. For example, in certain embodiments the range is between about 2 and 6 weeks, e.g., about 2 or 3 weeks. In one embodiment, the duration of the treatment cycle is 21 days (3 weeks). Within a treatment cycle, immunotoxin doses can be administered a various frequencies, and within a series of cycles, the duration of the cycles may differ. For example, doses can be administered every other day. In one embodiment, immunotoxin doses are administered, e.g., on days 1, 3, 5, 7, 9, and 11 of a 21 day treatment cycle.

Treatment can occur at regular intervals to prevent relapse, or upon indication of relapse. It will also be appreciated that the effective dosage of immunotoxin used for treatment can increase or decrease over the course of a particular treatment. Changes in dosage can result and become apparent from the results of diagnostic methods, for example, basic laboratory tests (e.g., peripheral blood smears to determine presence and morphology of blasts, cell counts, biochemical test to determine the presence or absence of various metabolic abnormalities and the degree of the abnormality, coagulation studies, etc.), immunophenotyping (e.g., morphologic, immunologic and genetic examination and classification), immunohistochemistry, cytogenetic and molecular diagnosis, minimal residues disease (MRD) studies, risk assessment studies, imaging (e.g., radiography, ultrasonography, echocardiography, etc.), etc. Diagnostic procedures can be performed using samples obtained from, e.g., peripheral blood, bone marrow aspirates, bone marrow biopsies, lumbar puncture, etc., prior, during, of after therapy.

In some embodiments of the invention, the patient is treated with escalating doses of the immunotoxin. Typically, patients receive an initial dose of immunotoxin, and the dose is escalated until the disease progresses or the toxicity becomes unacceptable.

In some cases, it is be desirable to use pharmaceutical compositions comprising the immunotoxin in an ex vivo manner. In such instance, cells, tissues, or organs that have been removed from the patient are exposed to the pharmaceutical compositions after which the cells, tissues or organs are subsequently implanted back into the patient.

In some embodiments of the present invention, the immunotoxin is administered in a dosage, such that an effective exposure is provided in a pediatric patient, for example as measured by, e.g., AUC, Cₘₐₓ, T_{1/2}, clearance, etc. The invention also includes methods which combine two or more favorable biological properties, such as pharmacokinetic parameters or combinations thereof. Examples of those pharmacokinetic parameters include arithmetic peak plasma concentration (Cₘₐₓ), biological half-life (T_{1/2}), arithmetic area under the curve from time zero to infinity (AUC_{0 ∞}), and clearance rate (Cl). For instance, the formulation administered through the methods of the invention can be selected such that when administered to a pediatric patient in need thereof, the selected formulation provides the pediatric patient with one or more of the desired pharmacokinetic parameters.

The biologically favorable property can be a desirable peak plasma concentration (Cₘₐₓ). In certain embodiments, the administration to a pediatric patient in need thereof achieves an arithmetic peak plasma concentration (Cₘₐₓ) of immunotoxin in a range of from about 311 ng/mL to about 586 ng/mL. In some embodiments, such Cₘₐₓ is achieved after a single dose of 30 μg/kg of immunotoxin by IV infusion over a period of about 30 minutes on the first day of the first treatment cycle.

In certain embodiments, peak plasma concentrations (Cₘₐₓ) of immunotoxin is higher than about 300 ng/mL. In some embodiments, such Cₘₐₓ value is reached after administration of a single dose of 30 μg/kg of immunotoxin by IV infusion over a period of about 30 minutes on the first day of the first treatment cycle.

In some embodiments, peak plasma concentrations (Cₘₐₓ) of immunotoxin is not lower than about 300 ng/mL, or not lower than about 275 ng/mL, or not lower than about 250 ng/mL, or not lower than about 225 ng/mL, or not lower than about 200 ng/mL, or not lower than about 175 ng/mL, or not lower than about 150 ng/mL, or not lower than about 125 ng/mL, or not lower than about 100 ng mL. In some embodiments, such Cₘₐₓ value is reached after administration of a single dose of 30 μg/kg of immunotoxin by IV infusion over a period of about 30 minutes on the first day of the first treatment cycle.

In yet further embodiments, the median of arithmetic peak plasma concentrations (Cₘₐₓ) of immunotoxin derived from a population of patients is greater than about 360 ng/mL. In some embodiments, such median Cₘₐₓ value is reached after administration of a single dose of 30 μg/kg of immunotoxin by IV infusion over a period of about 30 minutes on the first day of the first treatment cycle.

In some embodiments, the median of arithmetic peak plasma concentrations (Cₘₐₓ) of immunotoxin derived from a population of patients is about 300 ng/mL or greater, or about 320 ng/mL or greater, or about 340 ng/mL or greater, or about 360 ng/mL or greater, or about 380 ng/mL or greater, or about 400 ng/mL or greater; or about 420 ng/mL or greater; or about 440 ng/mL or greater; or about 460 ng/mL or greater; or about 480 ng/mL or greater; or about 500 ng/mL or greater. In some embodiments, such median value is reached after administration of a single dose of 30 μg/kg of immunotoxin by IV infusion over a period of about 30 minutes on the first day of the first treatment cycle.

In certain embodiments, the median of arithmetic peak plasma concentrations (Cₘₐₓ) of immunotoxin derived from a population of patients is about 516 ng/mL. In some embodiments, such median Cₘₐₓ value is reached after administration of a single dose of 30 μg/kg of immunotoxin by IV infusion over a period of about 30 minutes on the first day of the first treatment cycle.

In some embodiments, the biologically favorable property is a desirable biological (T_{1/2}). In some embodiments, the immunotoxin biological half-life (T_{1/2}) is in a range of from about 36 minutes to about 138 minutes. In some embodiments, such T_{1/2} value is reached after the administration of a single dose of 30 μg/kg of the immunotoxin by IV infusion over a period of about 30 minutes.

In some embodiments, the immunotoxin biological half-life (T_{1/2}) is not greater than 138 minutes. In some embodiments, such T_{1/2} value is reached after the administration of a single dose of 30 μg/kg of the immunotoxin by IV infusion over a period of about 30 minutes.

In some embodiments, the immunotoxin biological half-life (T_{1/2}) is lower than about 140 minutes, or lower than about 130 minutes, or lower than about 120 minutes, or lower than about 110 minutes, or lower than about 100 minutes, or lower than about 90 minutes, or lower than about 80 minutes, or lower than about 70 minutes, or lower than about 60 minutes, or lower than about 50 minutes, or lower than about 40 minutes. In some embodiments, such T_{1/2} value is reached after the administration of a single dose of 30 μg/kg of the immunotoxin by IV infusion over a period of about 30 minutes.

In yet further embodiments, the median of T_{1/2} values derived from a population of patients is lower than about 120 minutes. In some embodiments, such median T_{1/2} value is reached after the administration of a single dose of 30 μg/kg of the immunotoxin by IV infusion over a period of about 30 minutes.

In some embodiments, the median of T_{1/2} values derived from a population of patients is lower than about 110 minutes; or lower than about 100 minutes; or lower than about 90 minutes; or lower than about 80 minutes; or lower than about 70 minutes. In some embodiments, such median T_{1/2} value is reached after the administration of a single dose of 30 μg/kg of the immunotoxin by IV infusion over a period of about 30 minutes.

In some embodiments, the median of T_{1/2} values derived from a population of patients is about 60 minutes. In some embodiments, such median T_{1/2} value is reached after the administration of a single dose of 30 μg/kg of the immunotoxin by IV infusion over a period of about 30 minutes.

In some embodiments, the biologically favorable property is a desirable area under the curve from time zero to infinity (AUC_{0 ∞}). In one embodiment, a plot of the plasma concentration of immunotoxin versus time yields an arithmetic area under the curve from time zero to infinity (AUC_{0 ∞}) for immunotoxin in a range of from about 5.8 μg*min/mL to about 33.2 μg*min/mL. In some embodiments, such AUC_{0 ∞} value is reached following the administration of a single dose of 30 μg/kg of immunotoxin by IV infusion over a period of about 30 minutes.

In some embodiments, AUC_{0 ∞} values are not higher than about 33 μg*min/mL. In some embodiments, such AUC_{0 ∞} values are reached following the administration of a single dose of 30 μg/kg of immunotoxin by IV infusion over a period of about 30 minutes.

In yet further embodiments, the median of the AUC_{0 ∞} values derived from a population of patients is lower than about 50 μg*min/mL. In some embodiments, such median AUC_{0 ∞} values are reached following the administration of a single dose of 30 μg/kg of immunotoxin by IV infusion over a period of about 30 minutes.

In some embodiments, the median of the AUC_{0 ∞} values derived from a population of patients is lower than about 50 μg*min/mL; or lower than about 45 μg*min/mL; or lower than about 40 μg*min/mL; or lower than about 35 μg*min/mL; or lower than about 30 μg*min/mL; or lower than about 25 μg*min/mL; or lower than about 20 μg*min/mL; or lower than about 15 μg*min/mL, or lower than about 10 μg*min/mL. In some embodiments, such median AUC_{0 ∞} values are reached following the administration of a single dose of 30 μg/kg of immunotoxin by IV infusion over a period of about 30 minutes.

In some embodiments, the median of the AUC_{0 ∞} values derived from a population of patients is about 14.5 μg*min/mL. In some embodiments, such median AUC_{0 ∞} values are reached following the administration of a single dose of 30 μg/kg of immunotoxin by IV infusion over a period of about 30 minutes.

In certain embodiments, the biologically favorable property is a desirable clearance rate (Cl). In one embodiment, the immunotoxin clearance rate (Cl) is in a range from about 15,100 mL/kg/hour to about 85,200 mL/kg/hour. In some embodiments, such Cl value is reached following the administration of a single dose of 30 μg/kg of the immunotoxin by IV infusion over a period of about 30 minutes.

In some embodiments, the median of Cl values derived from a population of patients is about 36,400 mL/kg/hour. In some embodiments, such median Cl value is reached following the administration of a single dose of 30 μg/kg of the immunotoxin by IV infusion over a period of about 30 minutes.

In some embodiments, the median of Cl values derived from a population of patients is lower than about 90,000 mL/kg/hour, or lower than about 80,000 mL/kg/hour, or lower than about 70,000 mL/kg/hour, or lower than about 60,000 mL/kg/hour, or lower than about 50,000 mL/kg/hour, or lower than about 40,000 mL/kg/hour, or lower than about 35,000 mL/kg/hour, or lower than about 30,000 mL/kg/hour, or lower than about 25,000 mL/kg/hour, or lower than about 20,000 mL/kg/hour, or lower than about 15,000 mL/kg/hour. In some embodiments, such median Cl value is reached following the administration of a single dose of 30 μg/kg of the immunotoxin by IV infusion over a period of about 30 minutes.

In certain embodiments, the biologically favorable property is a desirable dissociation constant (K_{d}). In one embodiment, the immunotoxin has a binding affinity for CD22 with a dissociation constant (K_{d}) of less than 80 nM. If further embodiments, the Kd value is about 70 nM or lower, or about 60 nM or lower, or about 50 nM or lower, about 40 nM or lower, about 30 nM or lower, about 20 nM or lower, or about 10 nM or lower. In one embodiment, the immunotoxin has a binding affinity for CD22 with a dissociation constant (K_{d}) of about 6 nM.

The present invention provides methods for the treatment of ALL in pediatric patients wherein the inhibition of the growth of CD22-expressing (CD22+) cancer cells is caused by immunotoxin-induced cytotoxicity. In some embodiments, the immunotoxin-induced cytotoxicity causes an increase in cellular apoptosis. Upon binding to CD22, CAT-8015 is internalized and after processing, a portion of the toxin is transferred to the endoplasmic reticulum and translocated into the cytosol. In the cytosol, the toxin catalyses the ADP-ribosylation and inactivation of elongation factor-2, resulting in inhibition of protein synthesis and cell death.

Furthermore, the present invention provides methods for the treatment of ALL in pediatric patients wherein the in vitro cytoxicity of the CAT-8015 immunotoxin is greater than the level of cytotoxicity of the CAT-3888 immunotoxin, wherein the cytoxicity (LC₅₀) is measured as the concentration of immunotoxin that kills 50% of a population of viable B-lineage ALL cells. In certain embodiments, the ratio between the level of cytoxicity of the CAT-8015 immunotoxin and the level of cytotoxicity of the CAT-3888 immunotoxin, i.e., LC_{50 CAT-8015}/LC_{50 CAT-3888}, is at least 1.5. In other embodiments, the IC_{50 CAT-8015}/LC_{50 CAT-3888} ratio is about 2 or greater, or about 3 or greater, or about 4 or greater, or about 5 or greater, or about 6 or greater, or about 7 or greater, or about 8 or greater.

### Immunotoxin Formulations for the Treatment of Pediatric ALL

Formulations for the treatment of ALL are prepared for storage and use by combining the purified immunoconjugate with a pharmaceutically acceptable vehicle (e.g., carrier, excipient) (Remington, The Science and Practice of Pharmacy 20th Edition Mack Publishing, 2000). In some embodiments, the immunoconjugate is CAT-8015. Suitable pharmaceutically acceptable vehicles include, but are not limited to, nontoxic buffers such as phosphate, citrate, and other organic acids; salts such as sodium chloride; antioxidants including ascorbic acid and methionine; preservatives (e.g., octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes (e.g., Zn-protein complexes); and non-ionic surfactants such as TWEEN or polyethylene glycol (PEG).

CAT-8015 and CAT-8015 variants are useful for parenteral administration, such as intravenous administration or administration into a body cavity or lumen of an organ. The compositions for administration will commonly comprise a solution of CAT-8015 or variant dissolved in a pharmaceutically acceptable carrier, preferably an aqueous carrier. A variety of aqueous carriers can be used, e.g., buffered saline and the like. These solutions are sterile and generally free of undesirable matter. These compositions can be sterilized by conventional, well known sterilization techniques.

The compositions can contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of fusion protein in these formulations can vary widely, and will be selected primarily based on fluid volumes, viscosities, body weight and the like in accordance with the particular mode of administration selected and the patient's needs.

Controlled release parenteral formulations of the immunoconjugate compositions of the present invention can be made as implants, oily injections, or as particulate systems. For a broad overview of protein delivery systems see, Banga, A. J., “Therapeutic Peptides and Proteins: Formulation, Processing, and Delivery Systems” Technomic Publishing Company, Inc. 1995. Lancaster, Pa., incorporated herein by reference in its entirety.

Particulate systems include microspheres, microparticles, microcapsules, nanocapsules, nanospheres, and nanoparticles. Microcapsules contain the therapeutic protein as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously.

Microparticles are typically around 100 μm in diameter and are administered subcutaneously or intramuscularly. See, e.g., Kreuter, J. 1994. “Nanoparticles,” in Colloidal Drug Delivery Systems, J. Kreuter, ed., Marcel Dekker, Inc., New York, N.Y., pp. 219-342; Tice and Tabibi. 1992, “Parenteral Drug Delivery: Injectibles,” in Treatise on Controlled Drug Delivery, A. Kydonieus, ed., Marcel Dekker, Inc. New York, N.Y., pp. 315-339, each of which are incorporated herein by reference in its entirety.

Polymers can be used for use ion controlled release of immunoconjugate compositions of the present invention. Various degradable and nondegradable polymeric matrices for use in controlled drug delivery are known in the art. Langer, R. 1993. “Polymer-Controlled Drug Delivery Systems,” Accounts Chem. Res., 26:537-542. For example, the block copolymer, polaxamer 407 exists as a mobile viscous at low temperatures but forms a semisolid gel at body temperature. It has shown to be an efficacious vehicle for formulation and sustained delivery of recombinant interleukin-2 and urease. Johnston et al., Pharm. Res., 9:425-434 (1992); Pec et al., J. Parent. Sci. Tech., 44(2):58-65 (1990). Hydroxyapatite can also be used as a microcarrier for controlled release of proteins. Ijntema et al., Int. J. Pharm., 112:215-224 (1994). Liposomes can be used for controlled release as well as drug targeting of entrapped drug. Betageri et al., 1993. “Targeting of Liposomes,” in Liposome Drug Delivery Systems, Technomic Publishing Co., Inc., Lancaster, Pa.

Numerous additional systems for controlled delivery of therapeutic proteins are known. See, e.g., U.S. Pat. Nos. 5,055,303, 5,188,837, 4,235,871, 4,501,728, 4,837,028, 4,957,735 and 5,019,369; 5,055,303; 5,514,670; 5,413,797; 5,268,164; 5,004,697; 4,902,505; 5,506,206, 5,271,961; 5,254,342 and 5,534,496, each of which is incorporated herein by reference in its entirety.

In one embodiment, the immunoconjugate is formulated as a pharmaceutical composition comprising a pharmaceutically acceptable carrier. In another embodiment, the immunoconjugate is CAT-8015. In one embodiment, the immunoconjugate is a CAT-8015 variant. Pharmaceutically acceptable CAT-8015 immunoconjugate formulations include, but are not limited to:

| | | Molecular | | Quantity (per |
|---|---|---|---|---|
| Ingredient | Formula | Weight (Da) | Grade | liter) |
| Sodium Chloride | NaCl | 58.44 | USP | 29.22 g |
| Potassium | KH₂PO₄ | 136.09 | NF | 0.23 g |
| dihydrogen | | | | |
| phosphate | | | | |
| Disodium | Na₂HPO₄ | 141.96 | USP | 0.71 g |
| hydrogen | | | | |
| phosphate | | | | |
| Sodium hydroxide | NaOH | 40.00 | NF | As needed |
| WFI | H2O | 18.00 | USP | Qs, 1 L |

In one embodiment, the immunoconjugate is formulated as a pharmaceutical composition comprising at least one acceptable excipient. Pharmaceutically acceptable CAT-8015 (or CAT-8015 variant) immunoconjugate formulations include 0.5 mg/mL to 2.5 mg/mL CAT-8015, usually 1.0 mg/mL, 1.1 mg/mL, 1.2 mg mL, 1.3 mg/mL, 1.4 mg/mL or 1.5 mg/mL in 25 mM sodium phosphate, 4% sucrose, 8% glycine, 0.02% polysorbate 80 (PS80), pH 7.4. In additional embodiments, the sodium phosphate can be in a range of 20 mM to 100 mM, 25 mM to 50 mM, or 25 mM to 35 mM; the sucrose can be at 2%, 3%, 4%, 5% or 6%; the glycine can be in the range of 5-10%, usually, 5%, 6%, or 7% ; the polysorbate 80 can be in a range from about 0.01% to about 1%, usually 0.01%, 0.02%, 0.03%, 0.04% or 0.05%; with a pH in the range of 6.5 to 8.0, usually at pH 7.2, 7.3, 7.4, 7.5 or 7.6. Other buffering agents known to one of ordinary skill in the art can also be utilized.

In certain embodiments of the invention, the formulation is lyophilized. Lyophilized formulations or compositions are often made ready for use or reconstituted by addition of sterile distilled water. In certain embodiments, the lyophilized formulation of the invention is reconstituted into a vial.

For intravenous administration, a formulation of the invention, such as a liquid formulation or a formulation reconstituted from a lyophilized formulation is placed in a vial where the immunoconjugate in the formulation is present at concentrations as described above. This formulation is extracted from the vial and added to an intravenous (IV) bag solution, where the IV bag contains from about 30 mL to about 100 mL solution, usually 50 mL, 60 mL, 70 mL or 80 mL.

A separate IV bag of “protectant solution” can also be added to the total volume of the IV bag where the protectant solution contains polysorbate 80 in an amount such that the polysorbate 80 present in the final IV bag solution is in a range of 0.001% to about 3% polysorbate 80, usually in the range of about 0.01% to about 0.1%, and more usually at 0.01%, 0.02%, 0.03%, 0.04% or 0.05%. The protectant solution can be pre-formulated in a vial such that the polysorbate 80 is at a concentration of about 0.5% to about 5%, and can be 0.5%, 1.0%, 1.5%, 2.0%, 2.5%, 3.0%, 3.5%, 4.0%, 4.5% or 5.0% The protectant solution prevents adsorption of the immunoconjugate or drug (e.g., CAT-8015 or a CAT-8015 variant) to contact surfaces of the IV bag, thereby preventing or inhibiting the immunoconjugate or drug from sticking to the IV bag during administration and allowing the patient to receive the appropriate dosage of immunoconjugate or drug. The IV bag solution can be administered by infusion to the patient for various durations, usually 30 minutes to 1 hour, usually 30 minutes.

All documents, patents, journal articles and other materials cited in the present application are hereby incorporated by reference.

Although the present invention has been fully described in conjunction with several embodiments thereof with reference to the accompanying drawings, it is to be understood that various changes and modifications can be apparent to those skilled in the art. Such changes and modifications are to be understood as included within the scope of the present invention as defined by the appended claims, unless they depart there from.

### EXAMPLES

### Example 1

This Example sets forth the materials and methods in the studies reported in Example 2. See, Mussai F., et al., Br. J. Hematol. 150:352-358 (2010), which is hereby incorporated by reference in its entirety.

### ALL Samples

Blood and bone marrow samples were obtained from 35 patients with B-lineage ALL (Table SI) treated at the National Cancer Institute (NCI), St Jude Children's Research Hospital (SJCRH) or Johns Hopkins Hospital (JHH) with informed consent. The majority (n=22) were obtained from individuals with multiply relapsed ALL who were referred to the NCI for Phase I clinical trial participation. Thirteen patient samples from initial diagnosis were randomly selected from those available in the tumor banks at SJCRH and JHH. Thirty-three cases were characterized as pre-B ALL by flow cytometry and two as Burkitt-type/mature B-cell ALL. In all cases, >80% blasts expressed CD19 and CD22 antigens by flow cytometry. Cells were cryopreserved in RPMI-1640 medium with 10% fetal bovine serum (PBS) and 10% dimethyl sulfoxide. Institutional review board approval was obtained for the use of these samples in these studies.

### Culture Conditions

To measure the cytotoxic activity of the anti-CD22 immunotoxins CAT-8015 (HA22) and CAT-3888 (BL22), ALL cells were cultured on bone marrow-derived mesenchymal cells as previously described (Campana, et al., British J. Heamatol. 150:352-358 (1993)). Briefly, human bone marrow stromal cells that had been immortalized by telomerase transfection (developed at St Jude Children's Research Hospital) were cultured in RPMI-1640 medium (Invitrogen, Carlsbad, Calif., USA) with 10% fetal calf serum (Sigma-Aldrich, St Louis, Mo., USA). 2×10⁴ Stromal cells were plated into each well of flat-bottomed 96-well plates and cultured until confluent. On day 1 of the assay, ALL cells were resuspended in RPMI-1640 medium, 10% heat-inactivated FBS, glutamine (1×) and sodium pyruvate (1×). 3×10⁵ ALL cells were added to each well of stroma-coated plates. On day 2, CAT-3888 or CAT-8015 were added at final concentrations of 0, 0.5, 1, 5, 10, 50, 100 and 500 ng/ml in duplicate wells. SS1P (50 ng/mL), an antimesothelin Pseudomonas immunotoxin, was used as a negative control. The cytotoxicity of dexamethasone (10 μmol/L) was also tested in this assay. Cells were incubated for a further 72 h in a humidified atmosphere at 37° C. with 5% CO₂.

### Flow Cytometric Analysis

The cells were harvested by vigorous pipetting, transferred to Falcon tubes (Cat. No. 352052; BD Biosciences, San Jose, Calif., USA) and labelled with mouse anti-human CD19 antibody conjugated to fluorescein isothiocyanate (Cat. No. 555412; BD Pharmingen, San Jose, Calif., USA). The cells were resuspended in 1× Annexin Binding Buffer and labelled with 7-Aminoactinomycin D (7-AAD) and Annexin conjugated to phycoerythrin (PE) (Cat. No. 559763; PE Annexin V Apoptosis Detection kit I, BD Pharmingen). The cells were analysed with a FACScalibur flow cytometer in combination with CellQuest (Becton Dickinson, Franklin Lakes, N.J., USA) and FlowJo software (Tree Star Inc., Ashland, Oreg., USA).

Viability was assessed by setting gates based on the light scatter properties of the blasts. Due to spontaneous cell death, the relative percentage of viable cells at the end of the assay was calculated using the following formula: (no. of CD19⁺ viable cells recovered in test well/no, of CD19⁻ viable cells in untreated well×100). All results represent the mean of duplicate experiments.

The 50% lethal concentration (LC₅₀) was defined as the concentration of CAT-8015 that killed 50% of the viable cells at the termination of the assay.

Cell death by apoptosis was studied for all samples and late stage apoptosis was defined as cells positive for 7-AAD and Annexin-PE by flow cytometry. Non-apoptotic cells were those negative for 7-AAD and Annexin-PE.

Antigen site density was quantified by determining the anti-CD22 antibody binding capacity per cell (Schwartz et al., 1998). ALL samples were stained under saturating conditions with anti-CD22 antibody with 1:1 antibody to PE conjugation (BD Biosciences) using the BD Biosciences QuantiBRITE system for fluorescence quantitation. The antibody binding capacity value is the measurement of the mean value of the maximum capacity of each cell to bind the anti-CD22 antibody. QuantiBRITE beads are pre-calibrated standard beads containing known levels of PE molecules. QuantiBRITE beads were acquired on a FACSCalibur flow cytometer on the same day at the same instrument settings as the individual specimens. A standard curve comparing the geometric mean of fluorescence to known phycoerythrin content of the QuantiBRITE beads was constructed using QuantiCALC software.

### Immunotoxins and Chemotherapy Agents

The recombinant immunotoxins CAT-8015, CAT-3888 and SS1P were produced as previously described (Pastan, et al., Methods in Molecular Biology 248:503-518 (2004)). Dexamethasone was provided by the Division of Veterinary Resources (NIH).

### Statistical Analysis

An exact Wilcoxon rank sum test was used to determine the statistical significance of the difference in unpaired observations between two groups of pediatric patients. For comparison of LC₅₀ values between relapsed and newly diagnosed patients, an exact log-rank test was used because many of the observations were censored at CAT-8015 500 ng/ml concentration. A Wilcoxon signed rank test was used to determine whether the LC₅₀ ratios of CAT-3888/CAT-8015 were equal to 1.0. Correlations between parameters were evaluated using Spearman rank correlation analysis. All P-values are two-tailed and reported without adjustment for multiple comparisons. P<0.05 were considered to represent statistically significant effects.

### Example 2

The studies reported in this Example set out the results of in vitro cytotoxicity assays of CAT-8015 against pediatric ALL cells. These results demonstrate that the anti-CD22 immunotoxin CAT-8015, at concentrations achievable in patients, is highly cytotoxic to B-lineage ALL cells.

Summary: In vitro cytotoxicity of CAT-8015 against ALL blasts from newly diagnosed (n=13) and relapsed patients (n=22) was assessed using a bone marrow mesenchymal cell culture assay. There was interpatient variability in sensitivity to CAT-8015. Twenty-four of the 35 patient samples were sensitive (median 50% lethal concentration 3 ng/mL, range 1-80 ng/mL). Blasts from the other 11 patients were not killed by 500 ng/mL CAT-8015. The median 50% lethal concentration was 20 ng/mL for all patients. There was no significant difference in CAT-8015 sensitivity between diagnosis and relapse samples but peripheral blood ALL blasts were more sensitive to CAT-8015 that those from bone marrow (P=0.008).

### Cytotoxicity of CAT-8015 Against Pediatric ALL Cells

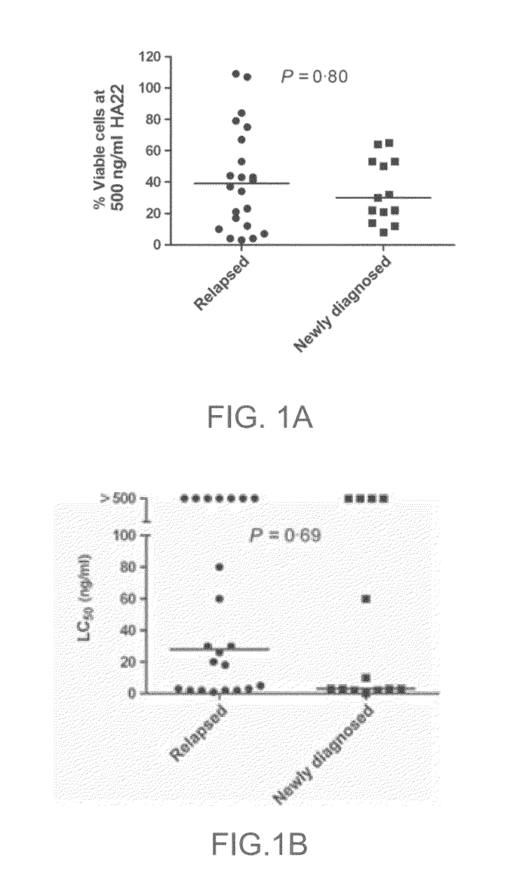

The cytotoxicity data of CAT-8015 against 35 cryopreserved patient samples is summarized in FIGS. 1A and 1B. Samples varied in their sensitivity, ranging from 100% cell death to almost complete resistance to killing at 500 ng/mL CAT-8015. Greater than 75% killing was achieved in 18 of 33 patient samples tested. There was no significant difference in CAT-8015 cytotoxocity (expressed as LC₅₀ or the percentage of viable cells after CAT-8015 treatment) when blasts from relapsed patients were compared to blasts from newly diagnosed patients (FIGS. 1A and 1B, P=0.69 and P=0.80, respectively).

Of the 22 relapse samples, eight were very sensitive to CAT-8015 with LC₅₀<=5 ng/mL, seven had a moderate response with LC₅₀s ranging from 18 to 80 ng/mL and seven samples were more resistant to CAT-8015 with 50% killing not achieved. Cells from 13 newly diagnosed patients also showed a range of sensitivity to CAT-8015. LC₅₀s ranged from 0.3 to 60 ng/mL (median 3 ng/mL) in nine samples, with seven showing extreme sensitivity (LC₅₀s≦3 ng/mL). Four samples appeared more resistant to CAT-8015 and 50% killing was not achieved at the 500 ng/mL concentration. The median LC₅₀ was 20 ng/mL for all patient samples tested.

### Relation Between In Vitro Sensitivity to CAT-8015 and Clinical and Cellular Features

There was no apparent association between CAT-8015 cytotoxicity and the age, sex, diagnostic white cell count or patient outcome following standard chemotherapy. The relationship between response to CAT-8015 and number CD22 sites per cell was investigated. CD22 site density, measured in 19 samples, ranged from 451 to 15,217 (median 4,063), and was only weakly correlated with CAT-8015-induced cytotoxicity (r=0.33, P=0,16) (FIG. 2). In contrast, the response to CAT-3888 appears to correlate with the number of CD22 sites per cell (Kreitman, R. J., et al., Clin. Cancer Res., 6:1476-1487 (2000) Kreitman, R. J., et al., Int. J. Cancer, 81:148-155 (1999)).

To assess whether the anatomical origin of ALL blasts might affect response to CAT-8015, samples derived from peripheral blood (n=9) and bone marrow (n=26) were compared for cytotoxicity. In general, peripheral blood ALL blasts were more sensitive to CAT-8015 than bone marrow blasts (3.6-fold difference in the percentage of viable cells, P=0.008).

### Specificity of CAT-8015 Cytotoxicity and Mechanism of Action

To ensure that the cytotoxicity observed was due to specific binding of CAT-8015 to CD22, an anti-mesothelin Pseudomonas immunotoxin SS1P was used as a negative control. SS1P at 500 ng/mL showed no cytotoxicity.

The activity of CAT-8015 to the lower affinity reagent CAT-3888 was also compared in a subset of samples found to be sensitive to CAT-8015. CAT-8015 was more active than CAT-3888 in all samples tested (TABLE I).

TABLE I
Cytotoxicity of BL22 and HA22 against patient samples.

| | |
|---|---|
| LC₅₀ (ng/ml) | LC₅₀ ratio (ng/ml) |

| Patient | BL22 | HA22 | BL22/HA22 |
|---|---|---|---|
| 5 | 20 | 3 | 6.7 |
| 9 | 3 | 2 | 1.5 |
| 10 | 3 | 2 | 1.5 |
| 20 | 20 | 5 | 4.0 |
| 22 | Not achieved | 60 | >8.3 |
| 29 | 1 | 0.3 | 3.4 |

LC₅₀, 50% lethal concentration

ALL cells were analysed for externalization of membrane phosphatidylserine and 7-AAD binding to measure apoptosis. In all cases CAT-8015 caused a dose-dependent increase in the percentage of cells undergoing apoptosis. There was close correlation of the percentage of viable ALL cells after treatment between light scatter properties and annexin/7-AAD staining for all samples.

### Cytotoxicity of CAT-8015 Compared to Dexamethasone

Dexamethasone, at a concentration of 10 μmol/l, had previously been shown to induce apoptosis in the majority of ALL patient samples cultured on stromal cells (Ito et al, J. Clin. Oncol. 14:2370-2376 (1996)). Different patterns of cytotoxicity were seen and patient samples showed a range of sensitivities to dexamethasone. The percentage of viable ALL cells at 10 μmol/l dexamethasone ranged from 4% to 158%, with a median of 40% (median of 29% for ALL samples from diagnosis and 42% for relapse samples, P=0.2). CAT-8015 was equally or more cytotoxic than dexamethasone in 17 patients. Resistance to CAT-8015 did not correlate with dexamethasone resistance (r=0.29, P=0.09). CAT-8015 could be cytotoxic to both dexamethasone-resistant and dexamethasone-sensitive ALL blasts in a dose-dependent manner. Resistance to dexamethasone correlated difference in sensitivity of blasts from blood or bone marrow to dexamethasone (P=0.27).

To confirm that CAT-8015 and dexamethasone were not cytotoxic to the stromal cells, confluent stromal cells were incubated with these agents for 72 h (500 ng/mL and 10 μmol/L, respectively). At the end of the assay, the stromal cells were incubated with WST-1. There was no difference in stromal cells incubated with CAT-8015 or dexamethasone compared to untreated controls. The stromal layers also remained intact without obvious morphological changes when microscopically examined.

This work establishes the mechanism of observed cytoxicity of CAT-8015. The majority of blasts were sensitive to CAT-8015, and cell death occurred via apoptosis. Furthermore, CAT-8015 was cytotoxic to relapsed, newly diagnosed, and dexamethasone-resistant patient samples. These data suggest that the mechanism of resistance to standard chemotherapy are different than those for PE-38 induced death.

CAT-8015 had greater activity than CAT-3888, e.g., demonstrating a 10-fold increase in the LC₅₀.

This study shows that CAT-8015 is cytotoxic to blasts from most patients and non-specific toxicities are expected to be less in comparison to chemotherapy. In vitro leukemia-stromal assays have been shown to be predictive of treatment outcome (Kumagai, M., et al., J. Clin. Inv., 97:755-760 (1996); Galderisi, F., et al., Pediatric Blood and Cancer, 53:543-550 (2009)).

### Example 3

### CAT-8015 (HA22) Phase I Clinical Trial

### Study Design

Selected Inclusion Criteria: Patients ≧6 months of age and <25 years of age with CD22+ B-lineage ALL or Non-Hodgkin Lymphoma (NHL) (≧30% abnormal cells as detected by fluorescence-activated cell sorting (FACS), ≧15% abnormal cells as detected by immunohistochemistry (IHC) relapsed or refractory to standard curative therapies were eligible for enrollment into the Phase I clinical trial.

Selection Exclusion criteria: Patients presenting isolated testicular or CNS disease were excluded. Also, patients with prior treatment with any Pseudomonas exotoxin compound were excluded from the trial.

Dosing: CAT-8015 (HA22) was administered at doses of 5 μg/kg, 10 μg/kg, 20 μg/kg, or 30 μg/kg ever-other-day for 6 doses every 21 days for up to 6 cycles. Doses were administered as 30 minute IV infusions.

Dose escalation phase: An accelerated dose escalation protocol was established wherein one patient was enrolled at each of the first 3 dose levels (5 μg/kg, 10 μg/kg, 20 μg/kg), with standard 3+3 dose escalation commencing at the 30 μg/kg dose. Patients received CAT-8015 until the disease progressed or toxicity was unacceptable.

All patients received acetaminophen, ranitidine, and diphenhydramine to mitigate infusion-related symptoms, and prophylaxis for central-nervous-system leukemia with intracethecal hydrocortisone, cytarabine, and methotrexate. Patients at high risk for tumor lysis syndrome received standard prophylaxis.

Disease Assessment and Response Criteria: CR (complete response) is defined as the attainment of an M1 (<5% blasts) bone marrow status on day 15 with no evidence of circulating blasts or extramedullary disease. PR (partial response) is defined as at least 50% decrease in the percentage of marrow blasts and achievement of an M2 (≦25% blasts) marrow status on day 15 with no evidence of circulating blasts or extramedullary disease. PD (progressive disease) or Relapse is defined as deterioration in marrow classification (i.e., M status) with at least a 50% increase in the percentage of marrow blasts compared to best response or no change in marrow classification (i.e., M status), but a 50% or greater increase in absolute peripheral blast count or extent of extramedullary disease compared to best response. A patient who fails to qualify as a CR, PR, HA or PD is defined as having stable disease (SD)

As Phase I studies are designed to evaluate toxicity and to define the maximum tolerable dose (MTD) and severe toxicities, they are not well suited to evaluate efficacy. However, surrogate endpoints can provide valuable insights into drug activity, mechanisms of action, and regimens to test in subsequent clinical trials that are designed to evaluate efficacy. To this end, the response category of “Hematological Activity” (HA) was used. HA is defined as not meeting the criteria for CR (complete response), PR (partial response) or PD (progressive disease), with any of the following: (i) at least a 50% decrease in the percentage of marrow blasts, (ii) at least a 50% decrease in the absolute peripheral blast count, or (iii) improvement of the ANC (absolute neutrophil count)≧1,000/μL or platelet count to ≧100,000/μL.

### Results

Seven pediatric patients with ALL (6 had precursor-B ALL, 1 had mature B-cell Burkitt-ALL), 5 to 17 years of age (median, 10) were treated on the clinical trial. All patients had been heavily pre-treated and had baseline cytopenias due to active malignancy and thus were not evaluable for hematological toxicities. The number of prior therapies ranged from 2 to 4 (median, 2). 6 patients were refractory to chemotherapy. 4 patients had undergone prior alloSCT (stem cell transplantation). One patient was male and six were females. The median ECOG status was 1.5 (1-2 range) and the Lansky status was 100 for all seven patients.

The most common adverse events observed to were hyperbilirubinemia, transaminase elevations, hyoalbuminemia, elevated creatinine, febrile bneutropenia, abdominal pain, pyrexia, hypertension, microscopic proteinuria, hemoglobinuria, hypoxia, and pleural effusion. Two of 4 patients treated at 30 μg/kg experienced Grade 3 or greater toxicity consistent with capillary leak: 1 with Grade 3 pleural effusion and hypoxia and 1 with Grade 4 vascular leak syndrome. All toxicities attributed to CAT-8015 were reversible.

Clinical activity was demonstrated in 4 of 7 pediatric patients. One 8 year old chemotherapy-refractory ALL patient treated at 10 μg/kg for 3 cycles had a complete remission (CR, complete response) by morphology and flow cytometry (a detailed description of the observed response in this patient is included in Example IV).

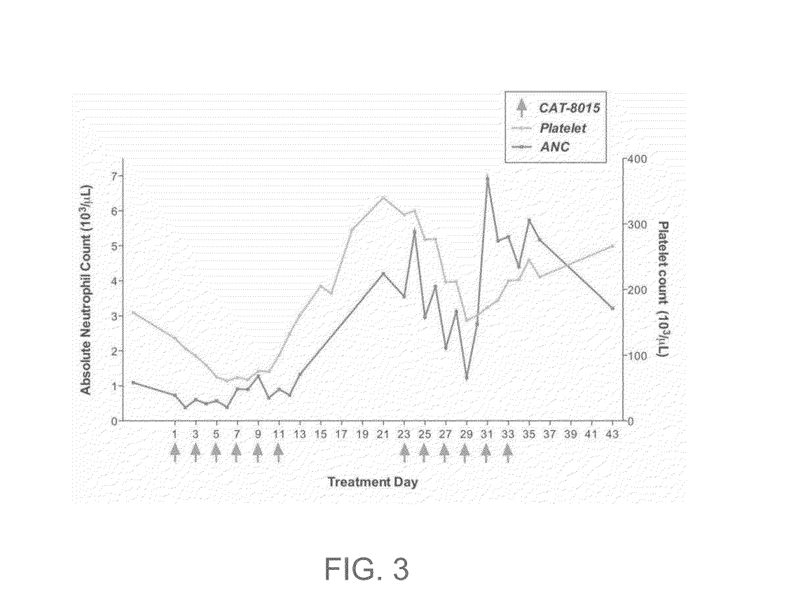

Three patients, who had received one or two cycles of treatment, met the protocol definition for hematological activity (blood count improvement). One of these patients developed high-titer neutralizing antibodies. FIG. 3 shows the blood counts in one of the pediatric patients treated with CAT-8015 who showed hematological activity after two treatment cycles with 30 μg/kg dosage. Counts were falling due to progressive bone marrow infiltration at the time of trial enrollment. There was normalization of the absolute neutrophil count (ANC) and platelet count with 1 cycle, followed by secondary decrease, and then improvement again with cycle 2.

Two patients, each treated for a single cycle, met the protocol definition for stable disease. The patient treated at the lowest dose level had progressive disease.

The pharmacokinetic parameters for the patients in the study are show in Table II.

TABLE II

| | | C_{MAX} | | AUC_{INF} | |
|---|---|---|---|---|---|
| Dose | Sub- | Day 1 | T_{1/2} | (mcg * | Clearance |
| (μg/kg) | jects | (ng/mL) | (hours) | hour/mL) | (mL/kg/hour) |
| 5 | 1 | BQL | — | — | — |
| 10 | 1 | 126 | — | — | — |
| 20 | 1 | 187 | 0.3 | 134 | 149,000 |
| 30 | 4* | 516 | 1.0 | 867 | 36,400 |
| | | (311-586) | (0.6-2.3) | (352-1,990) | (15,100-85,200) |

### Example 4

### CAT-8015 (HA22) Expanded Phase I Clinical Trial

The initial clinical trial described in Example 3 was expanded to include a total of 14 pediatric patients with ALL (12 evaluable for response). Patients were evaluated for response to CAT-8015 at different doses. 12 of 14 were refractory to prior chemotherapy regimens. The range of prior regimens was 2-7 (median 4). 7 patients had received prior stem cell transplantation.

Clinical activity was demonstrated in 8 of 12 evaluable pediatric patients (66%). Completed responses were observed in 3 patients (25%), and 5 other patients had hematologic improvement (41.6%).

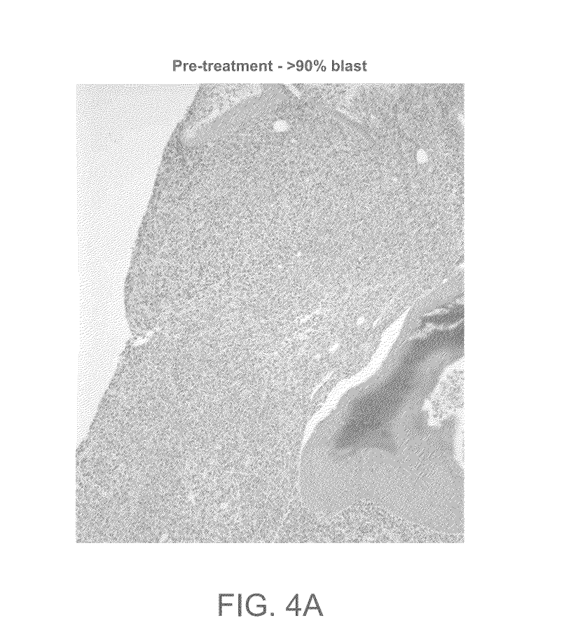

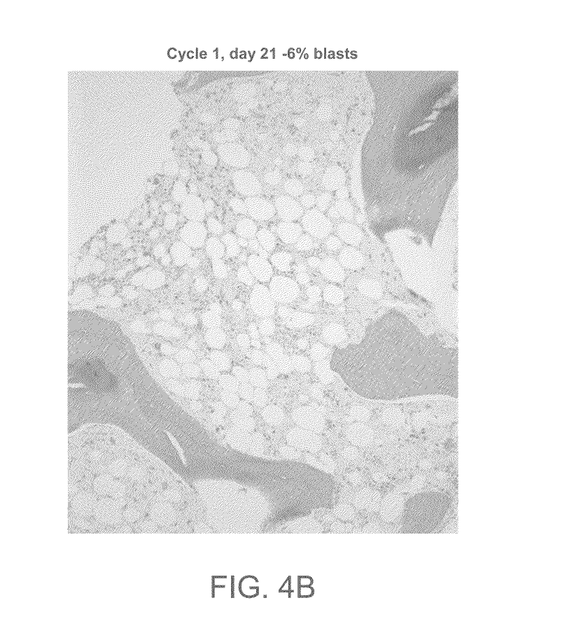

The first patient showing complete response after treatment with CAT-8015 was an 8 year old patient with chemotherapy-refractory ALL. This is the patient that was first identified as showing complete response in the initial phase I trial described in Example 3. The concentration of CD22 was approximately 15,000 sites per cell. After one 6-dose cycle of treatment with 10 μg/kg doses administered in alternate days, the percentage of blasts was reduced from over 90% prior to treatment to approximately 6% at the end of the treatment cycle, as shown in the hematoxylin and eosin bone marrow stains of FIGS. 4A and 4B. Morphologic Complete Remission (CR) was documented after 2 cycles. Flow cytometry Minimal Residual Disease (MRD) studies revealed 2.76% blasts after cycle 1 and 0.05% blasts after cycle 2.

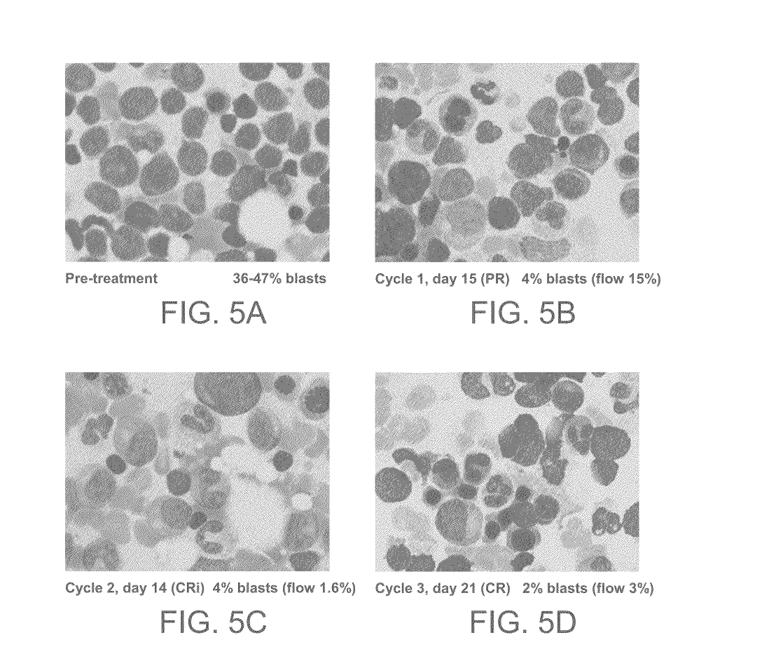

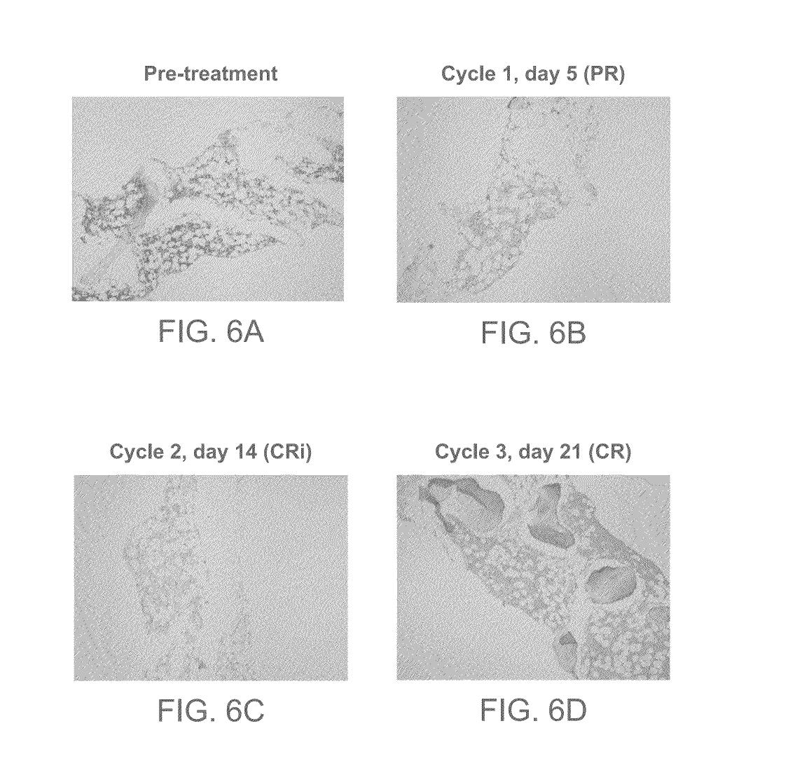

The second patient showing complete response after treatment with CAT-8015 was an 11 year old patient with multiply recurrent ALL who had undergone two prior stem cell transplants. The concentration CD22 was approximately 1,000 sites per cell. FIGS. 5A, 5B, 5C and 5D show Wright-Giemsa stains of bone marrow aspirates. FIGS. 6A, 6B, 6C and 6D show terminal deoxynucleotidyl transferase (TdT) stains of bone marrow biopsy samples, TdT is a unique enzyme that possesses the ability to add deoxynucleoside triphosphates to DNA without the use of template instruction. TdT expression has been reported to occur in over 90% of cases of ALL (Bolton, F. J., “The limited localization and conserved structure of TdT” in Leukemia Markers. Academic Press Inc., San Diego, 1981, pp. 33-40). TdT staining is found in all subtypes of ALL with the exception of pre-B-cell ALL (Srivasta, B., Leak Res. 4:209-215 (1980); Lanham, G. R., et al., Am. J. Clin. Pathol. 83:366-370 (1985)). After a 6-dose cycle of treatment with 20 μg/kg doses of CAT-8015, the percentage of blasts was reduced from 36-47% blasts prior to treatment, to 4% blast at day 14 of treatment cycle 1. Flow cytometry data indicated a reduction to 15% blasts. This reduction is considered a Partial Response. After a second cycle of treatment, cytology data still showed 4% blasts. However, flow cytometry data indicated a reduction to 1.6% blasts. After a third cycle of treatment, histochemistry data indicated that the percentage of blasts was reduced to 2%. Flow cytometry data showed the presence of 3% blasts. The reductions in blasts observed after treatment cycles 2 and 3 is considered a Complete Response.

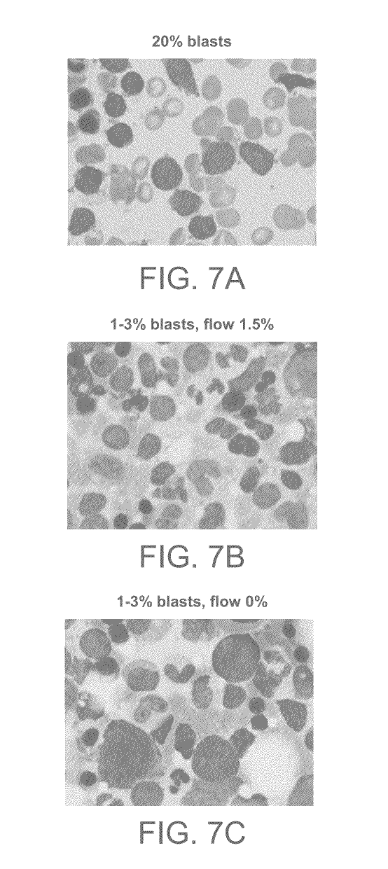

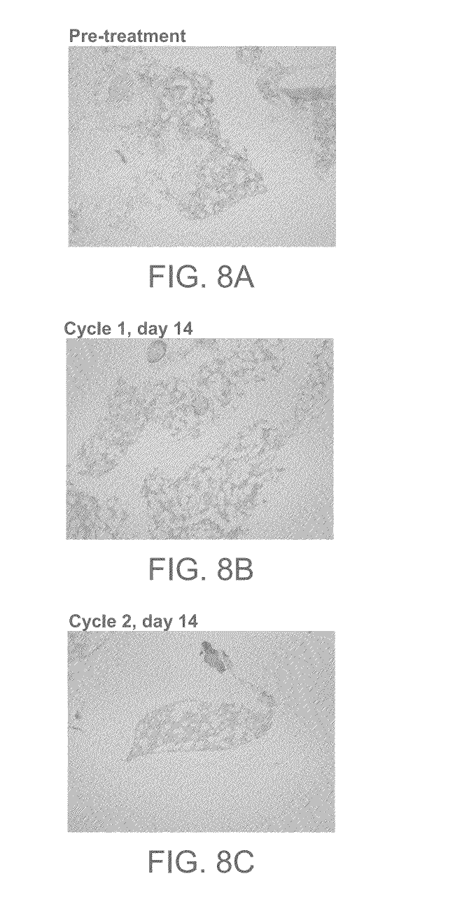

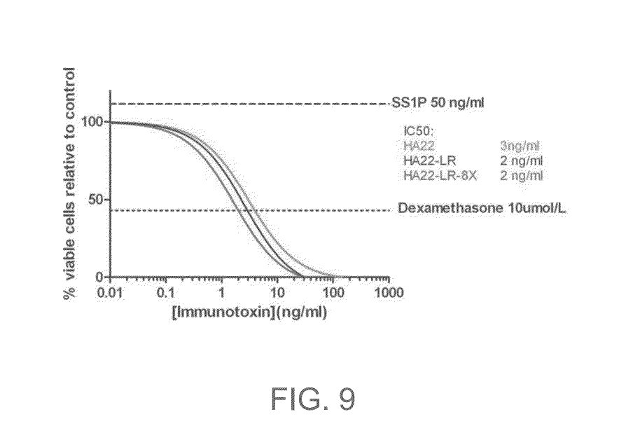

The third patient showing complete response after treatment with CAT-8015 was a 14 year old patient with chemotherapy-refractory ALL who had undergone a prior stem cell transplant. The concentration of CD22 was approximately 3,000 sites per cell. FIGS. 7A, 7B, 7C and 7D, and FIGS. 8A, 8B, 8C, and 8D show Wright-Giemsa and Tdt stains, respectively, as described above. After one 6-dose cycle of treatment with 30 μg/kg doses of CAT-8015, the percentage of blasts was reduced from 20% prior to treatment to 1-3% at day 14 of treatment cycle 1. Flow cytometry data indicated a reduction to 1.5% blasts. After a second cycle of treatment, the percentage of blasts was still 1-3%. However, flow cytometry data at day 14 of treatment cycle 2 showed no blasts (0%).

### Example 5

### Activity of CAT-8015 (HA22) Variants Against ALL Blast

In vitro data showed that CAT-8015 (HA22) variants where PE-LR or PE-LR-8X is the conjugated toxin, are highly active against ALL blasts (both cell lines and patient samples) in cytotoxicity assays.

Table III presents pre-clinical evidence of activity of CAT-8015 (HA22) and the mutants HA22-LR, HA22-LR-8X, and HA22-LR-KDEL against ALL blast cell lines.

TABLE III
Cell lines

| | | LC50 (ng/ml) | | |
|---|---|---|---|---|
| Cell line | Cell Type | HA22 | HA22-LR | HA22-LR-KDEL |
| NALM 6 | ALL | 5 | 1.5 | 1 |
| REH | ALL | 1 | 0.3 | 0.2 |
| KOPN 8 | ALL | 0.02 | 0.1 | 0.3 |
| SEM | ALL | 0.2 | 0.2 | 0.2 |
| EU | ALL | 0.5 | 1 | 0.3 |
| 697 | ALL | 0.5 | 0.4 | 0.2 |
| RAJI | Burkitt | 0.05 | 0.3 | 0.2 |
| CA46 | Burkitt | 0.2 | 0.7 | 0.3 |

| | | | LC50 (ng/ml) |
|---|---|---|---|
| | Cell Type | HA22 | HA22-LR-8X |
| NALM 6 | ALL | 3 | 2 |
| REH | ALL | 0.3 | 0.5 |
| KOPN 8 | ALL | 0.1 | 0.4 |
| SEM | ALL | 4 | 7 |
| EU | ALL | 0.4 | 0.3 |
| 697 | ALL | 2 | 1 |
| RAJI | Burkitt | 0.1 | 0.2 |
| CA46 | Burkitt | 0.4 | 0.6 |

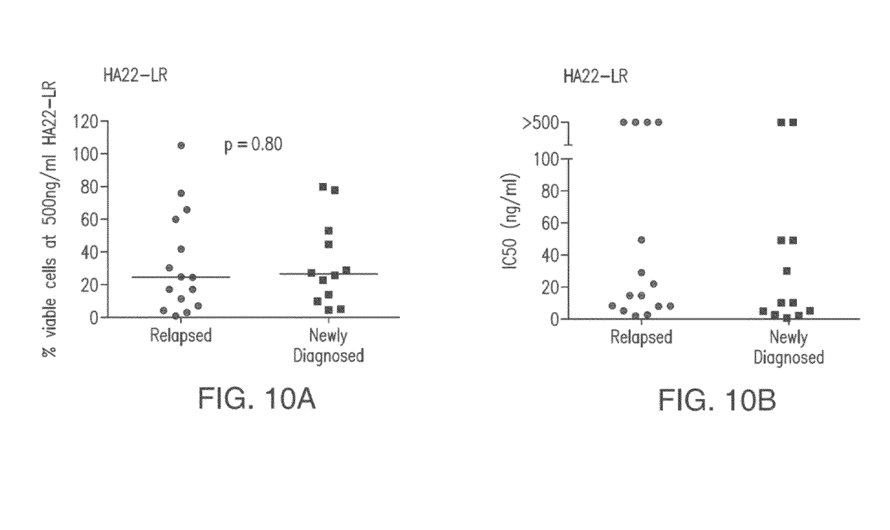

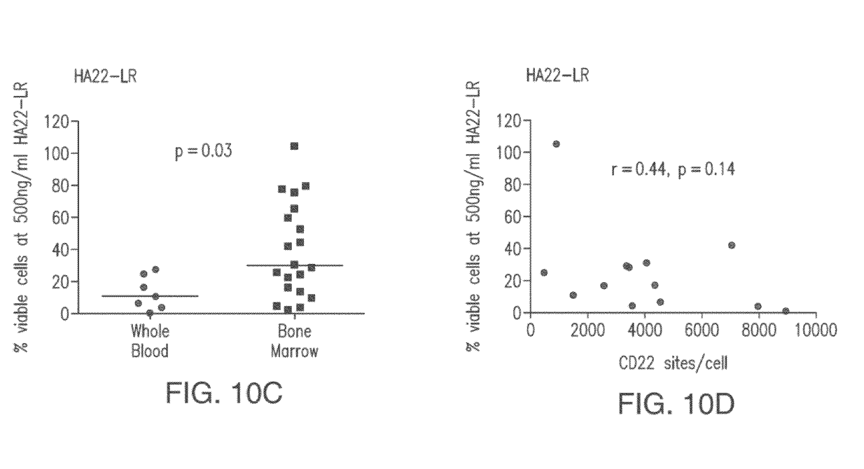

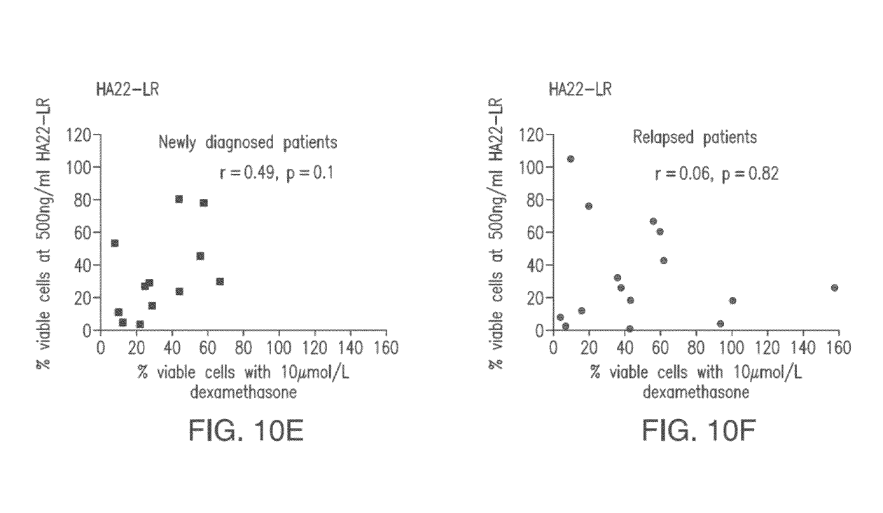

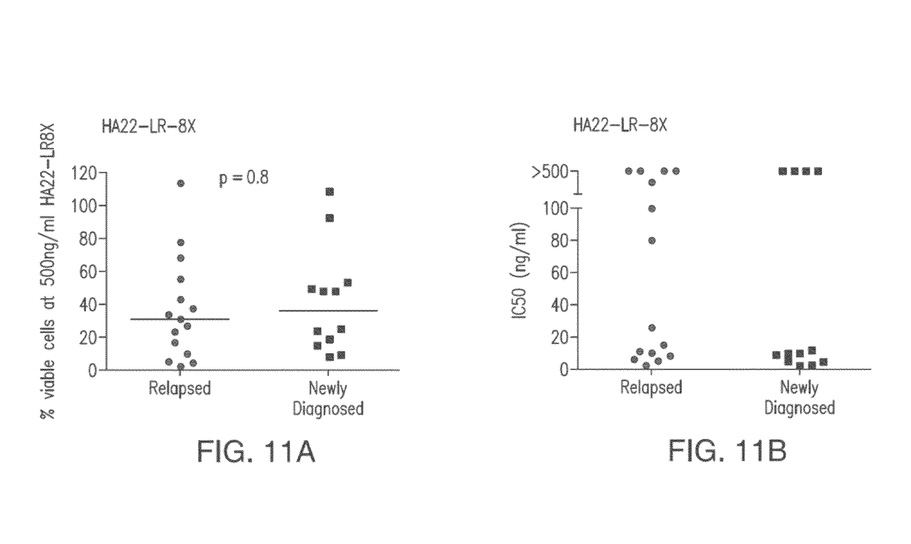

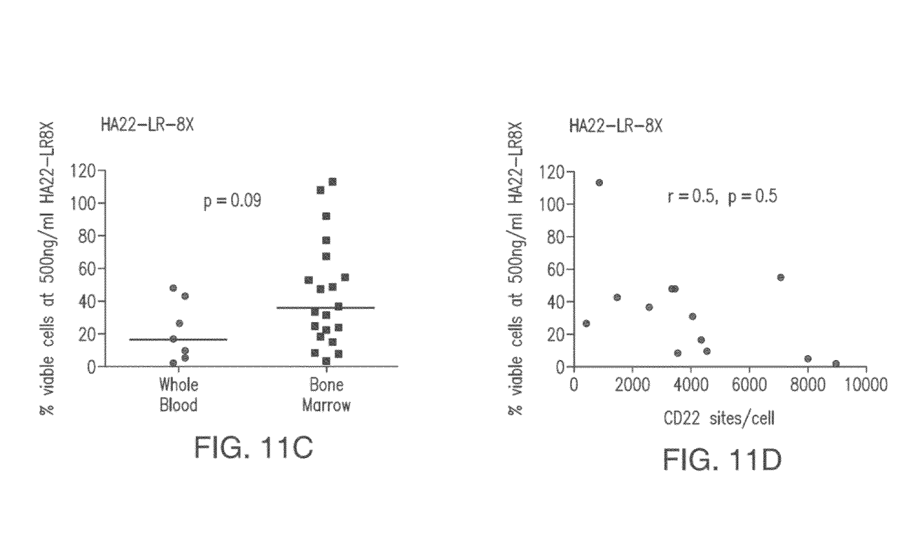

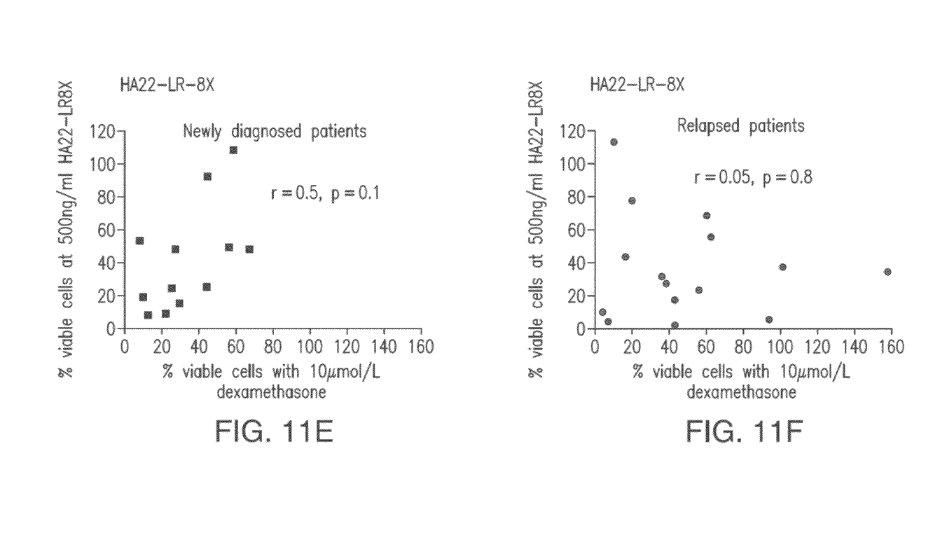

Cell-based assays performed on blast cells from pediatric ALL patients showed pre-clinical evidence of activity of the CAT-8015 (HA22) variants HA22-LR, and HA22-LR-8X (see FIGS. 10A, 10B, 10C, 10D, 10E, and 10F; and FIGS. 11A, 11B, 11C, 11D, 11E, and 11F, respectively).

1. A method of treating pediatric Acute Lymphoblastic Leukemia (ALL), comprising administering to a pediatric patient in need of said treatment an effective dose of a recombinant immunotoxin, wherein the immunotoxin comprises a variable light (V_{L}) chain comprising SEQ ID NO: 5 and a variable heavy (V_{H}) chain comprising SEQ ID NO: 1,
(i) wherein said V_{H} chain is genetically fused to a therapeutic moiety comprising a PE38 Pseudomonas exotoxin A fragment selected from the group consisting of SEQ ID NO: 2, SEQ ID NO: 6, SEQ ID NO: 7, and SEQ ID NO: 8;
(ii) wherein the recombinant immunotoxin specifically binds CD22 and inhibits the growth of CD22-expressing (CD22⁺) cancer cells, and wherein the inhibition is CD22-density independent and,
(iii) wherein said immunotoxin is administered to the pediatric patient in need of treatment at a dosage from 5 μg/kg to about 100 μg/kg.

2. The method of claim 1, wherein the antigen-binding portion of the immunotoxin is selected from the group consisting of a full antibody, an scFv, a dsFv, a Fab, and a F(ab′)₂.

3. The method of claim 1, further comprising a linker interposed between the variable heavy (V_{H}) chain and the therapeutic moiety.

4. The method of claim 3, where the linker comprises SEQ ID NO: 4.

5. (canceled)

6. The method of claim 1, wherein said variable heavy (V_{H}) chain-therapeutic fusion protein comprises SEQ ID NO:3.

7. The method of claim 1, wherein said immunotoxin comprises a dsFv, said dsFv comprising SEQ ID NO:1 and SEQ ID NO:5.

8. The method of claim 1, wherein said immunotoxin is CAT-8015.

9. The method of claim 1, wherein the patient suffers from ALL, relapsed ALL, or refractory ALL.

10. The method of claim 1, wherein the immunotoxin is administered in a combination therapy.

11. The method of claim 10, wherein the immunotoxin is administered to the patient during or after treatment with at least one single-agent or multi-agent combination treatment regimen.

12. The method of claim 10, wherein the immunotoxin is administered to a patient who has received a stem cell transplant or a bone marrow transplant prior to the treatment with the immunotoxin.

13. The method of claim 10, wherein the immunotoxin is administered to a patient who has received radiation therapy either as conditioning for bone marrow transplant or stem cell transplant or as a therapy.

14. The method of claim 1, wherein the immunotoxin is formulated with a pharmaceutically acceptable carrier, adjuvant, diluent, excipient, or any combinations thereof.

15. The method of claim 14, wherein the immunotoxin concentration is from about 0.5 mg/mL to about 2.5 mg/mL.

16. The method of claim 15, wherein the immunotoxin concentration is about 1 mg/mL, or about 1.1 mg/mL, or about 1.2 mg/mL, or about 1.3 mg/mL, or about 1.4 mg/mL, or about 1.5 mg/mL.

17. The method of claim 15, wherein the immunotoxin concentration is about 0.7 mg/mL.

18. The method of claim 14, wherein the immunotoxin is formulated as a solution for injection comprising sodium chloride, potassium dihydrogen phosphate, disodium hydrogen phosphate, and sodium hydroxide, wherein said immunoconjugate comprises a polypeptide comprising SEQ ID NO:3 and a polypeptide comprising SEQ ID NO:5.

19. The method of claim 10, wherein the combination therapy comprises the administration of at least one therapeutic agent select from the group consisting of an antibody or fragment thereof, a cytotoxic agent, a drug, a toxin, a radionuclide, an immunomodulator, a photoactive therapeutic agent, a radiosensitizing agent, and a hormone.

20. The method of claim 1, wherein the immunotoxin is administered as an intravenous injection.

21. The method of claim 20, wherein the intravenous injection is an intravenous infusion (IV infusion).

22. The method of claim 21, wherein the IV infusion is administered over a period of about 30 minutes.

23. The method of claim 1, wherein the inhibition of the growth of CD22-expressing (CD22⁺) cancer cells following the administration of the immunotoxin results in complete remission (complete response), improvement in response, lowering of leukemia burden, or a combination thereof.

24. The method of claim 1, wherein inhibition of the growth of CD22-expressing (CD22⁺) cancer cells tumor following the administration of the immunotoxin results in complete remission

25. (canceled)

26. The method of claim 1, wherein the immunotoxin dose is about 5 μg/kg, about 10 μg/kg, about 20 μg/kg, about 30 μg/kg, about 40 μg/kg, about 50 μg/kg, about 60 μg/kg, about 70 μg/kg, about 80 μg/kg, about 90 μg/kg, or about 100 μg/kg.

27. The method of claim 1, where the immunotoxin administered for one or more treatment cycles.

28. The method of claim 1, wherein the patient is treated with escalating doses of the immunotoxin.

29. The method of claim 1, wherein arithmetic peak plasma concentration (Cₘₐₓ) of immunotoxin is in a range of from about 311 ng/mL to about 586 ng/mL after the administration of a single 30 μg/kg dose of immunotoxin.

30. The method of claim 29, wherein the median of the arithmetic peak plasma concentrations (Cₘₐₓ) of immunotoxin derived from a population of patients is about 516 ng/mL.

31. The method of claim 29, wherein the median of arithmetic peak plasma concentrations (Cₘₐₓ) of immunotoxin derived from a population of patients is greater than about 360 ng/mL after the administration of a single 30 μg/kg dose of immunotoxin.

32. The method of claim 8, wherein the immunotoxin biological half-life (T_{1/2}) is in a range of from about 36 minutes to about 138 minutes.

33. The method of claim 32, wherein the median of T_{1/2} values derived from a population of patients is about 60 minutes.

34. The method of claim 32, wherein the median of T_{1/2} values derived from a population of patients is lower than about 100 minutes.

35. The method of claim 8, wherein a plot of the plasma concentration of immunotoxin versus time yields an arithmetic area under the curve from time zero to infinity (AUC_{0 ∞}) for immunotoxin in a range of from about 5.8 μg*min/mL to about 33.2 μg*min/mL after the administration of a single 30 μg/kg dose of immunotoxin.

36. The method of claim 35, wherein the median of the AUC_{0 ∞} values derived from a population of patients is about 14.5 μg*min/mL.

37. The method of claim 35, wherein the median of the AUC_{0 ∞} values derived from a population of patients is lower than about 50 μg*min/mL.

38. The method of claim 8, wherein the immunotoxin clearance rate (Cl) is in a range from about 15,100 mL/kg/hour to about 85,200 mL/kg/hour after the administration of a single 30 μg/kg dose of immunotoxin.

39. The method of claim 38, wherein the median of Cl values derived from a population of patients is about 36,400 mL/kg/hour.

40. The method of claim 29, comprising the administration of a single dose of 30 μg/kg of the immunotoxin by IV infusion over a period of about 30 minutes.

41. The method of claim 1, wherein the immunotoxin has a binding affinity for CD22 with a dissociation constant (K_{d}) of less than 80 nM.

42. The method of claim 41, wherein the immunotoxin has a binding affinity for CD22 with a dissociation constant (K_{d}) of about 6 nM.

43. The method of claim 1, wherein the growth inhibition is caused by immunotoxin-induced cytotoxicity.

44. The method of claim 43, wherein the immunotoxin-induced cytotoxicity causes an increase in cellular apoptosis.


SEQUENCE LISTING (8 sequences)

SEQ ID NO: 1 - protein, 123 aa, Mus musculus
  MISC_FEATURE: VH moiety of the VH-PE38 subunit of CAT-8015
MEVQLVESGGGLVKPGGSLKLSCAASGFAFSIYDMSWVRQTPEKCLEWVAYISSGGGTTYYPDTVKGRFTISRDNAKNTLYLQMSSLKSEDTAMYYCARHSGYGTHWGVLFAYWGQGTLVTVS

SEQ ID NO: 2 - protein, 347 aa, Pseudomonas aeruginosa
  MISC_FEATURE: Pseudomonas exotoxin PE38 moiety of the VH-PE38 subunit of CAT-8015
PEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 3 - protein, 476 aa, Mus musculus
  MISC_FEATURE: CAT-8015 VH-PE38 subunit comprising VH domain, linker, and PE38 domain
MEVQLVESGGGLVKPGGSLKLSCAASGFAFSIYDMSWVRQTPEKCLEWVAYISSGGGTTYYPDTVKGRFTISRDNAKNTLYLQMSSLKSEDTAMYYCARHSGYGTHWGVLFAYWGQGTLVTVSAKASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 4 - protein, 5 aa, Artificial Sequence
  Synthetic
KASGG

SEQ ID NO: 5 - protein, 108 aa, Mus musculus
  MISC_FEATURE: VL subunit of CAT-8015
MDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGCGTKLEIK

SEQ ID NO: 6 - protein, 230 aa, Artificial sequence
  Synthetic construct
RHRQPRGWEQLPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 7 - protein, 230 aa, Artificial sequence
  Synthetic construct
RHRQPRGWEQLPTGAEFLGDGGDVSFSTRGTQNWTVERLLQAHRQLEEGGYVFVGYHGTFLEAAQSIVFGGVRARSQDLDAIWAGFYIAGDPALAYGYAQDQEPDAAGRIRNGALLRVYVPRSSLPGFYATSLTLAAPEAAGEVERLIGHPLPLRLDAITGPEEAGGRLETILGWPLAERTVVIPSAIPTDPRNVGGDLDPSSIPDSEQAISALPDYASQPGKPPREDLK

SEQ ID NO: 8 - protein, 230 aa, Artificial sequence
  Synthetic construct
RHRQPRGWEQLPTGAEFLGDGGAVSFSTRGTQNWTVERLLQAHRQLEEGGYVFVGYHGTFLEAAQSIVFGGVRARSQDLDAIWAGFYIAGDPALAYGYAQDQEPDAAGAIRNGALLRVYVPRSSLPGFYATSLTLAAPEAAGEVERLIGHPLPLRLDAITGPEEAGGRLETILGWPLAERTVVIPSAIPTDPRNVGGDLDPSSIPDSEQAISALPDYASQPGKPPREDLK